(19) United States
(12) Patent Application Publication
(10) Pub. No.: US 2024/0336673 A1
(43) Pub. Date: Oct. 10, 2024

(54) MONOCLONAL ANTIBODIES TO PROTECT AGAINST GRAM-NEGATIVE PATHOGENS

(71) Applicant: WEST VIRGINIA UNIVERSITY BOARD OF GOVERNERS ON BEHALF OF WEST VIRGINIA UNIVERSITY, Morgantown, WV (US)

(72) Inventors: Mariette Barbier, Morgantown, WV (US); Fredric Heath Damron, Morgantown, WV (US); Jason Kang, Morgantown, WV (US); Margalida Mateu Borras, Morgantown, WV (US)

(21) Appl. No.: 18/627,809

(22) Filed: Apr. 5, 2024

Related U.S. Application Data
(60) Provisional application No. 63/494,990, filed on Apr. 7, 2023.

(51) Int. Cl. C07K 16/12; A61K 39/00; A61P 31/04

(52) U.S. Cl. CPC C07K 16/1214; A61P 31/04; A61K 2039/505; C07K 2317/31; C07K 2317/33; C07K 2317/734

(57) ABSTRACT

A monoclonal antibody against a Gram-negative bacterial pathogen has:
a first variable region with a heavy chain peptide with SEQ. ID No. 2 or SEQ. ID No. 6, or with CDRs from SEQ. ID No. 2 or SEQ. ID No. 6; and a light chain peptide with SEQ. ID No. 4 or SEQ. ID No. 8, or CDRs from SEQ. ID No. 4 or SEQ. ID No. 8; or
a second variable region with a heavy chain peptide with SEQ ID. No. 10, or with CDRs from SEQ. ID No. 10; and a light chain peptide with SEQ ID. No. 12, or CDRs from SEQ. ID No. 12. The monoclonal antibody with the first variable region is configured to bind to lipopolysaccharides of serogroup O5 strains of Pseudomonas aeruginosa. The monoclonal antibody with the second variable region is configured to bind to a flagellin protein of the Gram-negative bacterial pathogen.

### STATEMENT OF GOVERNMENT SUPPORT

This invention was made with government support under HDTRA1-21-1-0001 awarded by the Defense Threat Reduction Agency. The government has certain rights in the invention.

### SEQUENCE LISTING

The sequence listing of the present application is submitted electronically as an XML formatted sequence listing with a file name “WVU 3063 Sequence Listing,” with a creation date of Mar. 25, 2024, and a size of 11,903 bytes (11.6 KB). This sequence listing is herein incorporated by reference into the specification in its entirety.

### TECHNICAL FIELD

Various exemplary embodiments disclosed herein relate generally to antibodies against drug-resistant pathogens.

### BACKGROUND

Antimicrobial resistance (AMR) has become an important public health challenge. The misuse and abuse of antimicrobials have caused an exponential increase on the number of infections caused by AMR pathogens over the last decades, reaching an estimated 1,27 million deaths worldwide in 2019. If the rising problem of AMR are not confronted, infections caused by these pathogens are predicted to cause up to 10 million deaths a year and cost the global economy up to 100 trillion US dollars by 2050.

Because of the alarming number of infections caused by AMR pathogens, in 2017 the World Health Organization (WHO) published a list of pathogens, classified as critical, high and medium priority, to prioritize and stimulate the research and development of new prophylactic and therapeutic strategies. Among the pathogens included on this list, carbapenem-resistant Pseudomonas aeruginosa was given a “critical” status. P. aeruginosa is a Gram-negative opportunistic pathogen responsible of a wide range of severe infections particularly concerning in health care settings, especially in intensive care units (ICU) where represents 23% of all ICU-acquired infections. P. aeruginosa causes acute respiratory infections in immunocompromised patients as well as urinary tract, burn wound, keratitis and bloodstream infections, with a high rate of mortality. It is also the most common Gram-negative organism responsible for chronic respiratory infections in cystic fibrosis patients. P. aeruginosa represents one of the ESKAPE pathogens (Enterococcus faecium, Staphylococcus aureus, Klebsiella pneumoniae, Acinetobacter baumannii, Pseudomonas aeruginosa and Enterobacter spp.), acronym used to emphasize the ability of this pathogens to elude the action of the antibiotics. Those pathogens are the leading cause of life-threating healthcare-associated infections (HAIs) throughout the world, being multidrug resistant (MDR) P. aeruginosa responsible for 13-19% of HAIs each year in the US.

Pseudomonas aeruginosa is an ubiquitous, Gram-negative pathogen that thrives in many natural environments and habitats. As part of the pseudomonad genus, P. aeruginosa has minimal nutritional requirements and can persist on “high-touch” surfaces found in hospital rooms and medical equipment. Consequently, P. aeruginosa is one of the most common sources for nosocomial infections and can cause serious illness in immunocompromised patients, such as ventilator-associated pneumonia (VAP) and primary bloodstream infection. Among Gram-negative bacteria, P. aeruginosa is the most common cause of VAP, and second most common cause of primary bloodstream infections. The healthcare and socioeconomic burden from VAP and primary bloodstream infections are significant, and these complications are associated with increased mortality, greater hospital costs, and longer lengths of stay.

Although the selection of appropriate antibiotics is central in improving patient outcomes, the development of antibiotic resistance by P. aeruginosa has presented an enormous challenge for healthcare providers. P. aeruginosa can develop resistance to multiple classes of antibiotics through intrinsic and acquired mechanisms of resistance, which include multi-drug efflux pumps, antibiotic-modifying enzymes, and the horizontal transfer of mobile genetic elements. The prevalence of multidrug-resistant (MDR) P. aeruginosa (resistance to three or more drug classes) is alarmingly high, and national surveillance data estimates that 20% of P. aeruginosa isolates display multidrug resistance. In 2017, MDR P. aeruginosa infections were responsible for 32,600 hospitalizations, 2,700 deaths, and $767 million in attributable healthcare costs alone. As a result, the CDC has listed MDR P. aeruginosa as a serious public health threat that requires immediate action. Although there is a need for new therapeutic agents against MDR P. aeruginosa, there are few drugs in the developmental pipeline.

An important virulence factor in the pathogenesis of P. aeruginosa infections is lipopolysaccharide (LPS), which promotes evasion of host defenses and establishment of infection. LPS is also a potent stimulator of the innate immune system and can generate strong inflammatory responses. This host response is mediated by the activation of toll-like receptor 4 (TLR4), which triggers production of pro-inflammatory cytokines, such as IL-βB, IL-6 and TNF-α. However, if left unregulated, an excessive production of pro-inflammatory cytokines can eventually lead to severe inflammatory conditions, such as sepsis and multi-organ failure. The passive administration of monoclonal antibodies (mAbs) can provide rapid protection against P. aeruginosa infections, and is ideal for susceptible individuals who are immunocompromised or have acute exposure to the pathogen. Numerous pre-clinical studies have demonstrated that the administration of anti-LPS mAbs against the O-antigen of P. aeruginosa reduces bacterial burden and improves survival in multiple animal models of infection. In a phase IIa clinical trial with Panubacumab, an anti-LPS mAb against serotype O11, it was shown that adjunctive therapy with standard of care antibiotics improved clinical resolution of patients with confirmed O11 pneumonia. Though there are currently no FDA approved mAbs for P. aeruginosa infections, the promising outcomes of these pre-clinical and clinical studies support the further development of anti-LPS mAbs against P. aeruginosa.

ESKAPE pathogens such as P. aeruginosa are not the only AMR pathogens of concern. Bacteria from the genus Burkholderia are also notoriously resistant to antimicrobials. This genus encompasses many human pathogens such as the select agents Burkholderia pseudomallei and Burkholderia mallei, causative agents of glanders and melioidosis respectively, and the Burkholderia cepacia complex, responsible for pulmonary infections in cystic fibrosis patients. The expression of a modified lipopolysaccharide (LPS) and a variety of dug efflux pumps as well as the production of beta-lactamases and the expression of an altered DNA gyrase are some of the strategies developed by Burkholderia to evade antibiotics. Without a fast and appropriate antibiotic treatment, Burkholderia infections have a high rate of mortality, especially for B. pseudomallei infections, which can surpass 50%.

Therefore, new or complementary strategies are needed for infections difficult to treat with conventional antimicrobials. The use of monoclonal antibodies (mAbs) to treat infectious diseases could represent the future of antimicrobial therapy. The administration of serum as therapeutics or passive immunization was one of the main approaches to treat infectious diseases such as diphtheria or tetanus. Although successful in many cases, the toxicity associated to the administration of serum together with the success of antibiotics and vaccines waned its use. However, with the emergence of antibiotic resistant pathogens, the interest in antibodies has been renewed. The development of mAbs was possible after the discovery of the hybridoma technique in the early 1970s by Kilner and Milstein. Compared to polyclonal antisera, mAbs have higher specificity and lower variability, resulting in less side effects because of their little cross reactivity with the host cells and normal flora. Since the approval of the first mAb for use in humans in 1986, around 100 mAbs have been approved, mainly for autoimmune diseases and cancer. However, despite the success of antibodies in those areas, the development of mAbs to prevent and treat infectious diseases has been slow and most of the antibodies approved are for viral infections, mainly SARS-COV-2 and Ebola virus. To date, there are only 3 antibodies US FDA approved to treat bacterial infections, and none of them for AMR pathogens. One of the reasons is the cost of production compared with other antimicrobial agents.

In view of the foregoing, it would be desirable to develop improved treatments for infection by MDR P. aeruginosa and other drug-resistant pathogens.

### SUMMARY

In light of the present need for new therapies for infection by MDR P. aeruginosa and other drug-resistant pathogens, a brief summary of various exemplary embodiments is presented. Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

Various embodiments disclosed herein relate to a monoclonal antibody against a Gram-negative bacterial pathogen, with:
a first variable region with:
a heavy chain having a protein sequence of SEQ. ID No. 2 or SEQ. ID No. 6;
and
a light chain having a protein sequence of SEQ. ID No. 4 or SEQ. ID No. 8; or
a second variable region with:
a heavy chain having a protein sequence of SEQ ID. No. 10; and
a light chain having a protein sequence of SEQ ID. No. 12.

In various embodiments, the monoclonal antibody has the first variable region, and is configured to bind to lipopolysaccharides of serogroup O5 strains of P. aeruginosa.

In various embodiments, the monoclonal antibody has the second variable region, and is configured to bind to a flagellin protein of the Gram-negative bacterial pathogen. The Gram-negative bacterial pathogen may be P. aeruginosa, Burkholderia pseudomallei, Burkholderia thailandensis, Burkholderia cepacian, Burkholderia cenocepacia, Klebsiella pneumoniae, Serratia marcescens, Enterobacter cloacae, Salmonella enterica, Acinetobacter baumannii, Escherichia coli, and Bordetella pertussis.

Various embodiments disclosed herein relate to a bispecific antibody or antibody fragment against a Gram-negative bacterial pathogen, including:
a first variable region with:
a heavy chain from a first antibody against a lipopolysaccharide from the Gram-negative bacterial pathogen linked to a light chain from a second antibody; and
a heavy chain from the second antibody linked to a light chain from the first antibody against the lipopolysaccharide from the Gram-negative bacterial pathogen.

Various embodiments disclosed herein relate to a bispecific antibody or antibody fragment against a Gram-negative bacterial pathogen, including:
a first variable region with:
a heavy chain from a first antibody against a flagellin from the Gram-negative bacterial pathogen linked to a light chain from a second antibody; and
a heavy chain from the second antibody linked to a light chain from the first antibody against the flagellin from the Gram-negative bacterial pathogen.

In the bispecific antibody or antibody fragment, the first antibody may bind a first lipopolysaccharide epitope from the Gram-negative bacterial pathogen, and the second antibody may bind a second lipopolysaccharide epitope from the Gram-negative bacterial pathogen. In various embodiments, the first antibody may bind a lipopolysaccharide from the Gram-negative bacterial pathogen, and the second antibody may bind a flagellin protein.

Various embodiments relate to a bispecific antibody or antibody fragment against a Gram-negative bacterial pathogen, wherein the Gram-negative bacterial pathogen is a serogroup O5 strain of P. aeruginosa, and the bispecific antibody or antibody fragment comprises:
a heavy chain antibody protein of SEG ID No. 2 linked to a light chain antibody protein sequence of SEQ ID No.: 8, and
a heavy chain antibody protein of SEG ID No. 6 linked to a light chain antibody protein of SEQ ID No.: 4.

Various embodiments relate to a bispecific antibody or antibody fragment against a flagellin protein of a Gram-negative bacterial pathogen, wherein the bispecific antibody or antibody fragment comprises:
a heavy chain antibody protein of SEG ID No. 2 or SEQ ID No.: 6 linked to a light chain antibody protein sequence of SEQ ID No.: 12, and
a heavy chain antibody protein of SEG ID No. 10 linked to a light chain antibody protein of SEQ ID No.: 4 or SEQ ID No.: 8.

### BRIEF DESCRIPTION OF THE DRAWINGS

In order to better understand various exemplary embodiments, reference is made to the accompanying drawings, discussed below.

FIGS. 1A and 1B show that PAO1-PIAAMV convalescent mice are protected against lethal P. aeruginosa challenge. Six-week old, female CD-1 mice were challenged with a sub-lethal dose (1.35×10⁷ CFU) of PAO1-PIAAMV and allowed to convalesce. Mice were then challenged with a lethal dose (1.4×10⁸ CFU) of PAO1-PIA and euthanized at 16 h post-challenge. FIG. 1A shows that bacterial burden in the lung and nasal cavity was significantly reduced in PAO1-PIAAMV convalescent mice compared to the naïve group. FIG. 1B shows that serum titers were significantly increased in PAO1-PIAAMV convalescent mice compared to the naïve group. Differences in bacterial burden and serum titers were determined using a two-tailed student's t test, where ** P<0.01, *** P<0.001, and **** P<0.0001 compared to naïve mice.

FIG. 1C shows comparison of colony morphology between PAO1 cultured on PIA and PAO1 cultured on PIAAMV used for dose preparation. Growth of PAO1 on PIAAMV induced a mucoid colony morphology.

FIGS. 2A to 2F show generation and characterization of WVDC-0357 (also identified herein as S1F9) and WVDC-0496 (also identified herein as S3D4) mAbs. FIG. 2A shows that initial hybridoma supernatant was screened against PAO1 using ELISA and hybridoma clones WVDC-0357 (S1F9) and WVDC-0496 (S3D4) were selected based on positive binding to whole bacteria. FIG. 2B shows that FACS binding analysis demonstrates surface binding of P. aeruginosa by mAbs WVDC-0357 (SIF9) and WVDC-0496 (S3D4). FIGS. 2C and 2D show that ELISA against P. aeruginosa strains from different serogroups demonstrate that mAbs WVDC-0357 (S1F9) and WVDC-0496 (S3D4) bind to serogroup O5 strains. FIGS. 2E and 2F show identification of LPS as the target of mAb binding. Whole cell lysate of PAO1 treated with proteinase K (PK) or sodium periodate (NAIO₄) was prepared and immunoblotted with mAbs WVDC-0357 (S1F9) and WVDC-0496 (S3D4).

FIGS. 3A and 3B show a Western blot analysis against LPS transposon mutant. Whole cell lysates from PAO1 Washington and PAO1 tn::wbpC were prepared and blotted against mAbs WVDC-0357 (S1F9) (FIG. 3A) and WVDC-0496 (S3D4) (FIG. 3B). The gene wbp (′ is located in the O-antigen operon.

FIG. 4 shows antibacterial activity of mAbs WVDC-0357 (S1F9) and WVDC-0496 (S3D4). PAO1 bacteria was grown to mid-log phase growth and incubated with mAbs WVDC-0357 (S1F9) or WVDC-0496 (S3D4) before plating onto PIA plates. MAbs WVDC-0357 (S1F9) and WVDC-0496 (S3D4) demonstrate a dose-response killing of bacterium. Results are represented as the average from three independent experiments. Percent killing was calculated by normalizing the number of CFUs from each sample to the mean CFUs of the no antibody control. One-way ANOVA with Dunnett's multiple comparison test was performed for statistical analysis. ** P<0.01, **** P<0.0001 compared to isotype control.

FIGS. 5A to 5C show antibacterial activity of mAbs WVDC-0357 (S1F9) and WVDC-0496 (S3D4) in the presence of macrophages and complement. PAO1 was cultured to mid-log phase growth and incubated with mAb only (WVDC-0357 (SIF9) in FIG. 5B, WVDC-0496 (S3D4) in FIG. 5C, or isotype control in FIG. 5A), mAb+J774A. 1 macrophages, mAb+baby rabbit complement, or mAb+J774A.1 macrophages+baby rabbit complement. Samples were plated on PIA for CFU enumeration. Percent killing was calculated by normalizing the number of CFUs from each sample to the mean CFUs of the no mAb control. Differences in percent killing was calculated using one-way ANOVA with Dunnett's multiple comparison test. ** P<0.01 compared to mAb only group.

FIGS. 6A to 6F show MAbs WVDC-0357 (SIF9) and WVDC-0496 (S3D4) mediate bacterial agglutination. FIG. 6A shows that in transparent cuvette tubes, fresh growth cultures were prepared in lysogeny broth by the addition of an overnight culture of PAO1 and mAb WVDC-0357 (S1F9), mAb WVDC-0496 (S3D4), or an isotype control mAb at a final concentration of 100 μg/ml. Absorbance of growth cultures were measured over the course of 24 h. FIGS. 6B and 6C show that PAO1 was stained with BacLight Red stain, incubated with mAb WVDC-0357 (S1F9), mAb WVDC-0496 (S3D4), or an isotype control mAb, and mounted on glass slides for visualization with fluorescent microscopy. Random area images were taken and analyzed with an aggregation cluster pipeline in CellProfiler for number of aggregates per image and individual aggregate diameter. FIGS. 6D to 6F show representative fluorescent microscopy images from the isotype control, mAb WVDC-0357 (SIF9), and mAb WVDC-0496 (S3D4) treated BacLight-PAO1 slides. Differences in number of aggregates and individual aggregate diameter were calculated using a one-way ANOVA with Dunnett's multiple comparison test. *P<0.05, *** P<0.001 compared to isotype control.

FIGS. 7A to 7C show differences between static and mixed growth cultures. PAO1 growth cultures were incubated with an isotype control mAb, mAb WVDC-0357 (S1F9), or mAb WVDC-0496 (S3D4) for 24 h. FIG. 7A shows that, for mAbs WVDC-0357 (S1F9) and WVDC-0496 (S3D4), large differences in absorbance were observed between static and mixed cultures. FIG. 7B shows that these differences in absorbance were attributed to the presence of bacterial aggregates which were visually apparent in static cultures. FIG. 7B shows that, upon mixing, cultures appeared the same for mAb WVDC-0357 (SIF9), mAb WVDC-0496 (S3D4), and the isotype control mAb groups.

FIGS. 8A to 8C show that MAbs WVDC-0357 (S1F9) and WVDC-0496 (S3D4) protect mice against lethal P. aeruginosa bloodstream infection. FIG. 8A shows that nine-week old, female CD-1 mice were prophylactically administered an isotype control mAb, Pa WCV serum, mAb WVDC-0357 (S1F9), or mAb WVDC-0496 (S3D4), and after 12 h, mice were challenged with an IP dose of PAO1 (5×10⁵ CFU) and monitored for up to 96 h for survival. In addition, a set of mice were euthanized at 6 h post-challenge to look at bacterial burden in the blood, kidney, and spleen. FIG. 8B shows survival of mice represented as Kaplan-Meier curves. There were 5-10 mice in each group. Differences in survival were calculated using the log-rank test. **** P<0.0001 compared to isotype control. FIG. 8C shows bacterial burden at 6 h post-challenge. Blood, kidney, and spleen homogenate was serially diluted and plated on PIA to determine bacterial burden. Bacteria was detected at the limit of detection for Pa WCV serum, mAb WVDC-0357 (S1F9) and mAb WVDC-0496 (S3D4). Differences in CFUs were calculated using the Kruskal Wallis test with Dunn's multiple comparison. ** P<0.01 compared to isotype control.

FIG. 9A shows clinical scoring of mice after lethal bloodstream challenge. Mice were administered either an isotype control mAb, Pa WCV serum, mAb WVDC-0357 (S1F9), or mAb WVDC-0496 (S3D4). After 12 h, mice were IP challenged with a lethal dose of PAO1 and monitored for survival. Daily assessments of mice were performed taking into account appearance, activity, eye closure, respiration quality, body temperature, and body weight loss. For each category, mice were scored from 0-4, where 0 represented no symptoms and 4 represented the most severe phenotype.

FIGS. 9B and 9C rectal temperature and body weight of mice after lethal bloodstream challenge, respectively. Mice were administered either an isotype control mAb, Pa WCV serum, mAb WVDC-0357 (S1F9), or mAb WVDC-0496 (S3D4).

FIGS. 10A to 10F show the level of inflammatory cytokines and chemokines in serum following lethal bloodstream challenge. Nine-week old, female CD-1 mice were prophylactically administered an isotype control mAb, Pa WCV serum, mAb WVDC-0357 (SIF9), or mAb WVDC-0496 (S3D4), and after 12 h were challenged with an IP dose of PAO1 (5×10⁵ CFU). Mice were euthanized at 6 h post-challenge, and the concentration of CXCL-1 (FIG. 10A), IFN-γ (FIG. 10B), TNF-α (FIG. 10C), IL-βB (FIG. 10D), IL-6 (FIG. 10E), and IL-10 (FIG. 10F) in the serum was quantified with the Luminex assay. Differences in cytokines and chemokines were calculated using the Kruskal Wallis test with Dunn's multiple comparison. *P<0.05, ** P<0.01, *** P<0.001 compared to isotype control.

FIG. 11 shows the pharmacokinetics of mAbs WVDC-0357 (S1F9) and WVDC-0496 (S3D4) in mice. Six-week old, female CD-1 mice were administered an isotype control mAb, mAb WVDC-0357 (S1F9), or mAb WVDC-0496 (S3D4) at 45 mg/kg. Mice were submandibular bled over the course of 21 d to assess serum IgG titers.

FIGS. 12A to 12D show that MAbs WVDC-0357 (SIF9) and WVDC-0496 (S3D4) increase bacterial clearance in an acute pneumonia model of infection. FIG. 12A shows that six week old, female CD-1 mice were prophylactically administered an isotype control mAb, Pa WCV serum, mAb WVDC-0357 (S1F9), or mAb WVDC-0496 (S3D4), and after 12 h were challenged with an intranasal dose of PAO1 (2×10⁷ CFU). Mice were euthanized at 16 h post-challenge to look at correlates of protection. FIGS. 12B and 12C show that a) bacterial burden in the lung and nasal wash and b) lung weights were significantly reduced for mAb WVDC-0357 (S1F9) and WVDC-0496 (S3D4) treated mice, respectively. FIG. 12D shows that rectal temperature was significantly reduced below physiological temperature in the isotype control treated mice. Differences in CFUs, lung weight, and temperature were calculated using a one-way ANOVA with Dunnett's multiple comparison test. * P<0.05, ** P<0.01, *** P<0.001, **** P<0.0001 compared to isotype control.

FIGS. 13A to 13F show the level of inflammatory cytokines and chemokines in the lungs. Six week old, female CD-1 mice were prophylactically administered an isotype control mAb, Pa WCV serum, mAb WVDC-0357 (S1F9), or mAb WVDC-0496 (S3D4), and challenged with an intranasal dose of PAO1 (2×10⁷ CFU). Mice were euthanized 16 h post-challenge and the concentration of CXCL-1 (FIG. 10A), IFN-γ (FIG. 10B), TNF-α (FIG. 10C), IL-βB (FIG. 10D), IL-6 (FIG. 10E), and IL-10 (FIG. 10F) in the lung supernatant was quantified using the Luminex assay. Differences in cytokines and chemokines were calculated using a one-way ANOVA with Dunnett's multiple comparison test. * P<0.05, ** P<0.01, *** P<0.001, **** P<0.0001 compared to isotype control.

FIGS. 14A to 14C show multi-antigen hybridoma screening by ELISA coated with FliC peptides. FIG. 14A shows ELISA screening with purified FliC protein from P. aeruginosa.

FIG. 14B shows ELISA screening with whole cell P. aeruginosa PAO1. FIG. 14C shows ELISA screening with whole cell B. pseudomallei Bp82.

FIG. 15 shows that WVDC-2109 binds to a protein on P. aeruginosa surface and not to a polysaccharide. Whole-cell lysates of P. aeruginosa PAO1 were treated with proteinase K (PK) or sodium periodate and immunoblotted with WVDC-2109. The MW lane is a molecular weight marker.

FIGS. 16A and 16B show that WVDC-2109 binds to P. aeruginosa and B. pseudomallei FliC. FIG. 16A shows an immunoblot using WVDC-2109 against 3 μg of recombinant FliC purified from P. aeruginosa or B. pseudomallei Bp82. FIG. 16B shows an immunoblot against 10 μg of P. aeruginosa PAO1 and B. pseudomallei Bp82 cell lysates using WVDC-2109 antibody. All blots in this panel were performed in parallel. Mw, Molecular weight size.

FIGS. 17A to 17D show that WVDC-2109 interacts with FliC without blocking TLR5 activation. FIG. 17A shows a model of the interaction between P. aeruginosa FliC and the predicted structure of WVDC-2109. FIG. 17B shows a model of the interaction between B. pseudomallei FliC and the predicted structure of WVDC-2109. FIG. 17C shows HEK-Blue™ hTLR5 cells stimulated with P. aeruginosa flagellin (0-40 ng/ml) previously incubated with increasing concentrations of WVDC-2109 (0-100 μg/ml). FIG. 17D shows HEK-Blue™ hTLR5 cells stimulated with B. pseudomallei flagellin (0-20 ng/ml) previously incubated with increasing concentrations of WVDC-2109 (0-100 μg/ml). SEAP activity was determined with HEK-Blue™ Detection media at 650 nm. Bar graphs represent mean+SD.

FIG. 18 shows that WVDC-2109 does not increase bacterial attachment-invasion to J774A. 1 cells. Bacterial attachment and invasion of P. aeruginosa PAO1 was determined by incubating J774A.1 cells and bacteria after opsonization with or without WVDC-2109. Statistical significance was determined by Student's t test. Dotted line indicates lowest limit of detection. Each dot represents one replicate. Error bars represent standard error of the mean.

FIG. 19 shows that WVDC-2109 does not kill P. aeruginosa PAO1. Bacterial killing was determined after 60 min of incubation with WCDC-2109. Statistical significance was determined by Student's t test. Dotted line indicates lowest limit of detection. Each dot represents one replicate. Error bars represent standard error of the mean.

FIGS. 20A and 20B show that WVDC-2109 increases bacterial killing mediated by complement system and opsonophagocytic killing by macrophages. FIG. 20A shows a complement bactericidal assay, giving the percentage of survival of P. aeruginosa PAO1 after 90 min of incubation with Heat-Inactivated (HI) Guinea Pig Complement, Guinea Pig Complement alone or Guinea Pig Complement+WVDC-2109. FIG. 20B shows opsonophagocytic killing by macrophage J774A. 1 cells of P. aeruginosa PAO1 previously opsonized with or without WVDC-2109. Statistical significance was determined by one-way ANOVA in (A) or by Student's t test in (B): * p<0.05, **** p<0.0001. Dotted line indicates lowest limit of detection. Each dot represents one replicate. Error bars represent standard error of the mean.

FIGS. 21A to 21C show survival of mice (FIG. 21A) and rectal temperatures (FIGS. 21B and 21C) over time of mice challenged with P. aeruginosa in a murine lethal model of sepsis. n=5 mice/group. Mice received anti-FliC mAb (FIG. 21A [blue line]; FIG. 22C) or an isotype IgG control (FIG. 21A [black line]; FIG. 22B) 12 h before P. aeruginosa PAO1 intraperitoneal infection. Statistical significance in FIG. 21A was determined using a Mantel-Cox test.

FIGS. 22A and 22B show that WVDC-2109 reduces bacterial burden in blood, kidney, and spleen. Mice received WVDC-2109 or an isotype IgG control 12 h before P. aeruginosa lethal infection. P. aeruginosa PAO1 presence in the blood, kidney, and spleen was determined after 6 h (FIG. 22A) and 12 h (FIG. 22B) of infection using serial dilutions. Each dot represents an individual animal. Experiments were performed with n=5 mice/group. Dotted line indicates lowest limit of detection.

FIGS. 23A to 23F show that TNFα, IL-βB, IL-6, IL-10 and IFN-γ are decreased in passive immunized WVDC-2109 mice. Levels of CXCL-1 (FIG. 23A), TNFα (FIG. 23B), IL-βB (FIG. 23C), IL-6 (FIG. 23D), IL-10 (FIG. 23E), and IFN-γ (FIG. 23F) in the serum of CD-1 mice after 6 and 12 h of P. aeruginosa lethal infection are shown. Mice were passively immunized with WVDC-2109 or with a non-specific IgG as a control 12 h before a P. aeruginosa PAO1 intraperitoneal infection.

FIGS. 24A to 24D show that WVDC-2109 reduces bacterial burden and lung edema during acute murine pneumonia. Bacterial burden in the respiratory tract 16 hours after P. aeruginosa challenge in CD-1 mice is shown. Mice received WVDC-2109, a non-specific isotype IgG control or serum from mice previously vaccinated with inactivated P. aeruginosa (Pa WCV) 12 h before P. aeruginosa PAO1 infection. Viable bacteria were quantified in the lung (FIG. 24A) and nasal wash (FIG. 24B) using serial dilutions. Rectal temperature (FIG. 24C) and lung weight (FIG. 24D) were also determined at time of euthanasia. Each dot represents an individual animal. Statistical significance was determined by one way ANOVA. * p<0.05, ** p<0.005, *** p<0.0005. Dotted line indicates lowest limit of detection.

FIGS. 25A to 25F show that WVDC-2109 reduces lung inflammation during acute murine pneumonia. CXCL-1 (FIG. 25A), TNFα (FIG. 25B), IL-βB (FIG. 25C), IL-6 (FIG. 25D), IL-10 (FIG. 25E), and IFN-γ (FIG. 25F) were quantified in the lung homogenate of CD-1 mice after 16 h of P. aeruginosa PAO1 intranasal infection. Mice were passively immunized with WVDC-2109, with a non-specific isotype IgG as a negative control or serum from mice previously vaccinated with inactivated P. aeruginosa (Pa WCV) 12 h before P. aeruginosa PAO1 infection. Each dot represents an individual animal. Statistical significance was determined by one way ANOVA. * p<0.05, ** p<0.005. Dotted line indicates lowest limit of detection.

FIGS. 26A to 26C show that WVDC-2109 is a cross-reactive antibody. FIG. 26A shows a Western Blot using WVDC-2109 against 10 μg of whole cell lysates from P. aeruginosa PAO1, P. aeruginosa PAK and P. aeruginosa clinical isolates (CEC 31, 32, 34, 38, 44, 45, 55, 60, 65, 75 and 86). FIG. 26B shows a Western blot against 10 μg of B. pseudomallei, B. thailandensis, B. cepacia and B. cenocepacia cell lysates using WVDC-2109. All blots in this panel were performed in parallel. FIG. 26C shows ELISA screening using WVDC-2109 against whole K. pneumoniae, S. marcescens, E. clocae, S. enterica, A. baumannii, E. coli, B. pertussis and P. aeruginosa.

FIGS. 27A and 27B show construction of bispecific antibodies by DART technology.

### DETAILED DESCRIPTION

P. aeruginosa Lipopolysaccharide (Lps) Antibodies

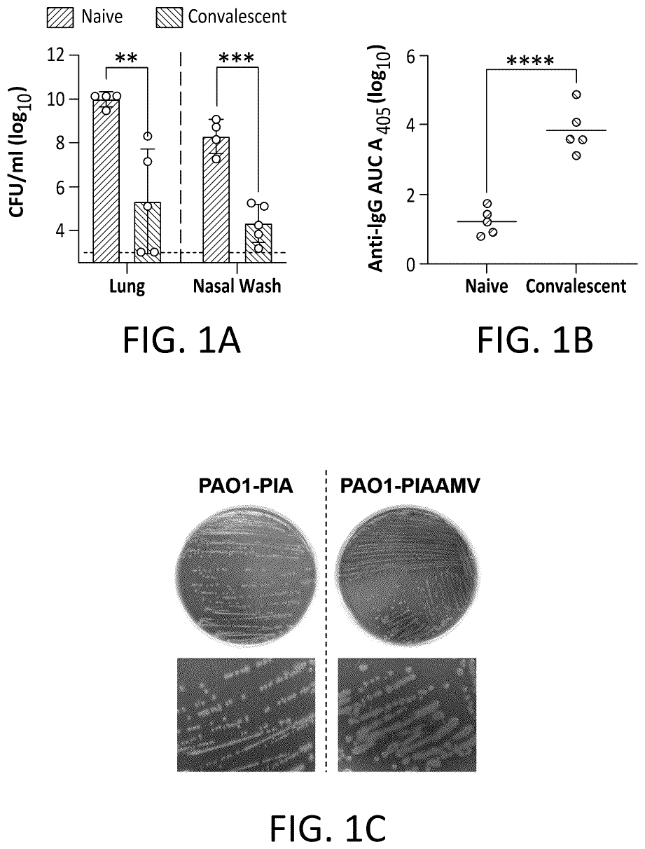

One method for upregulating bacterial cell surface polysaccharides is to use ammonium metavanadate (AMV), which is a phosphatase inhibitor that induces cell envelope stress responses. When P. aeruginosa is cultured on Pseudomonas isolation agar supplemented with AMV (PIAAMV), numerous genotypic changes occur, and the resultant phenotype closely resembles other clinical isolates that have undergone similar envelope stress responses. When P. aeruginosa reference strain PAO1 is cultured on PIAAMV, a large number of genes associated with LPS increased expression levels, including the entire O-antigen operon (wzz to wbpM). In addition, the lipid A moiety was altered to include a palmitate group, which is a common modification among cystic fibrosis isolates. As shown in FIG. 1C, the colony morphology of PAO1 cultured on PIA is different from PAO1 cultured on PIAAMV used for dose preparation. Growth of PAO1 on PIAAMV induced a mucoid colony morphology. In a survival study of mice infected with PAO1 cultured on PIA or PIAAMV, there was an improved survival in the PAO1-PIAAMV group. Therefore, PAO1 cultured on PIAAMV was used for generation of P. aeruginosa antibodies trough hybridoma technology. DNA, RNA, and peptide sequences disclosed herein were obtained from nucleic acids and peptides from such hybridomas.

Various embodiments disclosed herein relate to mAbs that are highly protective against P. aeruginosa infections. MAb WVDC-0357 (S1F9) (also known as WVDC-0357) and mAb WVDC-0496 (S3D4) (also known as WVDC-0496) were derived from mice infected with PAO1 cultured on PIAAMV. Such mAbs are highly protective in clinically relevant models of sepsis and acute pneumonia. MAbs WVDC-0357 (S1F9) and WVDC-0496 (S3D4) reduce the number of viable bacteria and protect against P. aeruginosa challenge in multiple models of infection. Furthermore, this protective effect was linked with a decreased inflammatory cytokine profile. Altogether, these results support the translation and use of mAbs WVDC-0357 (S1F9) and WVDC-0496 (S3D4) against P. aeruginosa infections.

### PAO1-PIAAMV Convalescent Mice are Protected in an Acute Murine Pneumonia Model.

Mice previously infected with PAO1-PIAAMV may be protected from a lethal P. aeruginosa infection. PAO1 was cultured on PIAAMV and a sub-lethal dose prepared (1.35×10⁷ CFU). Six-week old female CD-1 mice were challenged by the intranasal route and allowed to convalesce for 35 d. Naïve and convalescent mice were then challenged by the intranasal route with a lethal dose of PAO1 grown on PIA (1.4×10⁸ CFU). Mice were euthanized at 16 h post-challenge to determine bacterial burden in the lungs and nasal cavities.

FIG. 1A and FIG. 1B show that there were significant differences in bacterial burden between naïve and convalescent challenged mice. FIG. 1A shows that, when compared to naïve mice, convalescent mice had a 4.68 log reduction in CFUs for the lungs (P<0.01) and a 3.98 log reduction in CFUs for the nasal wash (P<0.001). FIG. 1B shows that significant differences were also found in serum antibody titers, as convalescent mice had 2.62 log increase (P<0.0001) in anti-IgG antibodies. Overall, this data demonstrates that PAO1-PIAAMV convalescent mice were protected against lethal challenge with one facet of protection conferred through antibody-mediated responses. Based on these findings, PAO1-PIAAMV was utilized as an immunization strategy to induce robust antibody-secreting cells for hybridoma generation.

Generation and Characterization of Monoclonal Antibodies Against P. aeruginosa

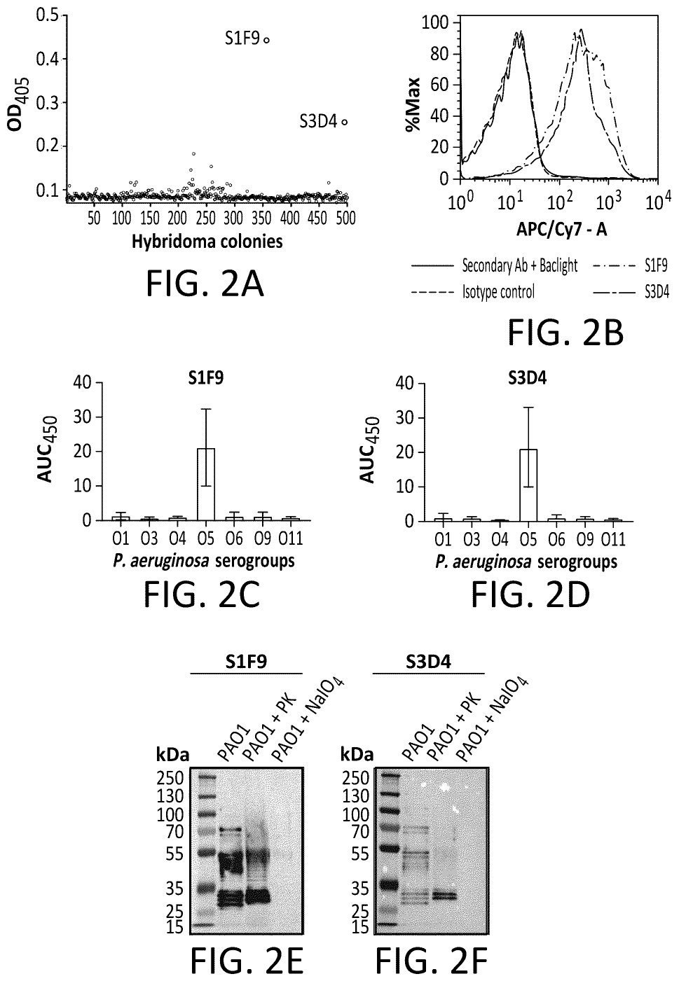

To generate mAbs against P. aeruginosa, CD-1 mice were immunized and boosted with live preparations of P. aeruginosa PAO1 grown on PIAAMV and following convalescence, hybridomas were formed by fusing spleen cells with immortalized myeloma cells. As shown in FIG. 2A, 508 hybridoma colonies were screened, and two IgG2b-kappa secreting hybridomas (WVDC-0357 (S1F9) and WVDC-0496 (S3D4)) that had strong binding to intact PAO1 by ELISA were identified. MAbs WVDC-0357 (SIF9) and WVDC-0496 (S3D4) were subsequently purified for further analysis. Flow cytometry was performed to confirm binding of mAbs WVDC-0357 (S1F9) and WVDC-0496 (S3D4) to intact PAO1, which demonstrated an approximate 1-log shift in fluorescence above the isotype-matched control, as shown in FIG. 2B. Sequencing of the antibody variable region revealed that mAbs WVDC-0357 (SIF9) and WVDC-0496 (S3D4) share similar variable regions with point mutations encoded in LFR3 and LFR4. For both antibodies, the light chain sequence was derived from the murine germline sequences IGKV1-122 and IGKJ2, and the heavy chain sequence was derived from the murine germline sequences IGHV1-52, IGHV1-61, IGHD1-1, and IGHJ4.

Referring to Table 1, the heavy chain antibody DNA sequences of mAb WVDC-0357 (S1F9) and mAb WVDC-0496 (S3D4) have SEQ. ID Nos.: 1 and 5, respectively. The heavy chain antibody protein sequences of mAb WVDC-0357 (S1F9) and mAb WVDC-0496 (S3D4) have SEQ. ID Nos.: 2 and 6, respectively; the complementarity-defining regions (CDRs) are shown in bold underlined text. Framework regions are shown in bold italic text. The light chain antibody DNA sequences of mAb WVDC-0357 (S1F9) and mAb WVDC-0496 (S3D4) have SEQ. ID Nos.: 3 and 7, respectively; again, the CDRs are shown in bold underlined text and framework regions are shown in bold italic text. The light chain antibody protein sequences of mAb WVDC-0357 (S1F9) and mAb WVDC-0496 (S3D4) have SEQ. ID Nos.: 4 and 8, respectively. Table 1a identifies the portions of SEQ ID Nos.: 2, 4, 6, and 8 corresponding to the various CDRs. The CDRs are hypervariable domains that determine specific antibody binding to, in this case, a lipopolysaccharide.

To further characterize the binding of mAbs WVDC-0357 (S1F9) and WVDC-0496 (S3D4), an ELISA screen was performed against a panel of P. aeruginosa strains, shown in Table 2. It was found that these mAbs bind to P. aeruginosa strains from serogroup 05, as shown in FIGS. 2C and 2B.

TABLE 2

| Bacterial Species | Source | Serogroup | Notes |
|---|---|---|---|
| P. aeruginosa PAO1 | M. Vasil | O5 | Laboratory strain |
| P. aeruginosa PAK | | | Laboratory strain |
| P. aeruginosa | ATCC | | |
| | 15442 | | |
| P. aeruginosa CEC31 | Burns, et | O9 | Collected from bronchioalveolar lavage, |
| | al. 2001 | | serotype O9, motile |
| P. aeruginosa CEC32 | Burns, et | O5 | Collected from oropharyngeal swab, |
| | al. 2001 | | serotype O5, motile |
| P. aeruginosa CEC34 | Burns, et | O1 | |
| | al. 2001 | | |
| P. aeruginosa CEC38 | Burns, et | | Collected from oropharyngeal swab, |
| | al. 2001 | | serotype O6, motile |
| P. aeruginosa CEC44 | Burns, et | O1 | Collected from oropharyngeal swab, |
| | al. 2001 | | serotype O1, motile |
| P. aeruginosa CEC45 | Burns, et | O6 | Collected from oropharyngeal swab, |
| | al. 2001 | | serotype O6, non-motile |
| P. aeruginosa CEC55 | Burns, et | O11 | Collected from oropharyngeal swab, |
| | al. 2001 | | serotype O11, non-motile |
| P. aeruginosa CEC60 | Burns, et | O6 | Collected from oropharyngeal swab, |
| | al. 2001 | | serotype O6, motile |
| P. aeruginosa CEC65 | Burns, et | | Collected from oropharyngeal swab, |
| | al. 2001 | | serotype O6, motile |
| P. aeruginosa CEC75 | Burns, et | O4 | Collected from bronchioalveolar lavage, |
| | al. 2001 | | serotype O4, motile |
| P. aeruginosa CEC79 | Burns, et | O5 | Collected from oropharyngeal swab, |
| | al. 2001 | | serotype O5, motile |
| P. aeruginosa CEC86 | Burns, et | O3 | Collected from oropharyngeal swab, |
| | al. 2001 | | serotype O3, motile |
| P. aeruginosa PAO1 | Held, et | O5 | |
| Washington | al. 2012 | | |
| P. aeruginosa PAO1 | Held, et | O5 | |
| tn::wbpC | al. 2012 | | |
| B. pseudomallei Bp82 | | | Avirulent B. pseudomallei 1026b ΔpurM |
| B. thailandensis E264 | | | |
| B. cepacia | ATTC | | |
| | 25416 | | |
| B. cenocepacia | K562 | | |
| Klebsiella pneumoniae | ATCC | | |
| | 13383 | | |
| Escherichia coli | ATCC | | |
| | 8739 | | |
| Acinetobacter | ATCC | | |
| baumannii | 17972 | | |
| Enterobacter clocae | ATCC | | |
| | BAA- | | |
| | 2468 | | |
| Salmonella enterica | ATCC | | |
| | 14028 | | |
| B. pertussis UT25Sm1 | Brickman | | |
| | T J et al, | | |
| | 1996 | | |

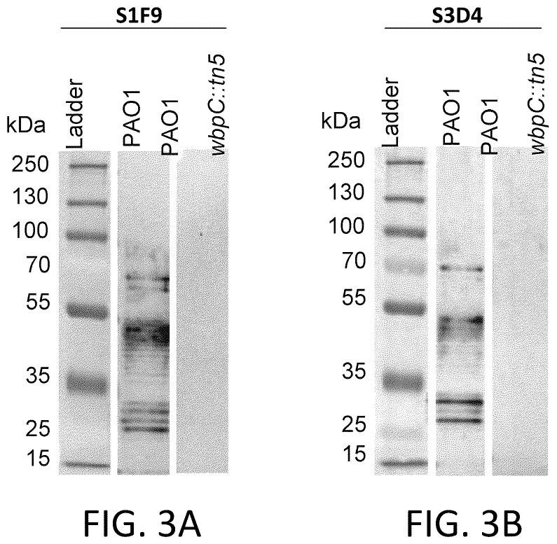

Based on the specificity of mAbs WVDC-0357 (S1F9) and WVDC-0496 (S3D4) to serogroup O5 strains, it was likely that these mAbs were directed against LPS. To confirm LPS as the target of binding, a western blot was performed with PAO1 cell lysates, including lysates treated with proteinase K (to digest proteins) and sodium periodate (to degrade polysaccharides). Western blot analysis revealed that the banding pattern by mAbs WVDC-0357 (SIF9) and WVDC-0496 (S3D4) against the PAO1 cell lysate was indicative of LPS as the antigen, as shown in FIGS. 2E and 2F. Furthermore, treatment of the PAO1 lysate with proteinase K did not alter binding; however treatment of the PAO1 lysate with sodium periodate completely ablated binding, indicative of a polysaccharide, rather than a protein-based target. Western blotting was also performed with a PAO1 transposon mutant strain deficient in the gene wbpC, which is located in the O-antigen biosynthesis cluster and functions as a possible O-acetyltransferase. MAbs WVDC-0357 (SIF9) and WVDC-0496 (S3D4) did not bind to the PAO1 tn::wbp (′ strain, as shown in FIG. 3. Collectively, these results indicate that mAb WVDC-0357 (SIF9) and WVDC-0496 (S3D4) bind to the O-antigen of LPS from serogroup 05.

MAbs WVDC-0357 (S1F9) and WVDC-0496 (S3D4) Mediate Direct Antibacterial Activity Against P. aeruginosa.

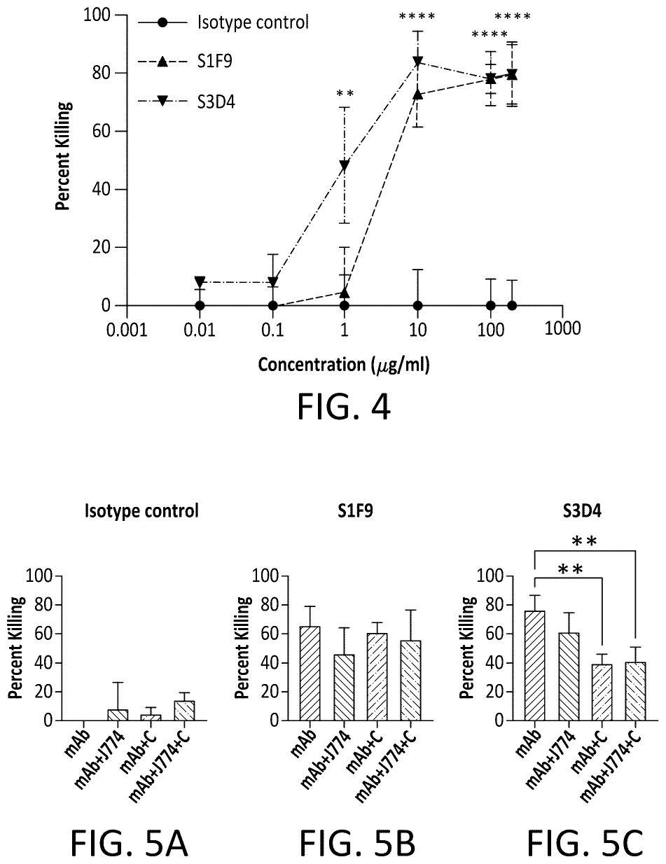

Antibodies display a wide and diverse set of functions, yet recent years have demonstrated the importance of the non-canonical function of antibodies in host immunity. Besides the effector system pathways, antibodies are able to mediate antimicrobial activity through mechanisms such as direct pathogen inactivation and agglutination. To determine the antibacterial activity of mAbs WVDC-0357 (SIF9) and WVDC-0496 (S3D4), bacterial killing assays were performed, whereby mid-log phase PAO1 was incubated with mAb WVDC-0357 (SIF9), mAb WVDC-0496 (S3D4), or an isotype control mAb, and plated for CFU enumeration. FIG. 4 shows that MAbs WVDC-0357 (SIF9) and WVDC-0496 (S3D4) displayed a dose-dependent killing of PAO1 with around a 70-80% reduction in viable bacteria at concentrations greater than 10 μg/ml. To determine the role of immune cells in facilitating mAbs WVDC-0357 (WVDC-0357 (SIF9)) and WVDC-0496 (S3D4) to clear bacteria, the same assay was performed with the addition of J774A.1 murine macrophages and baby rabbit complement, however there was no significant increase in bacterial killing with the addition of either component, as shown in FIG. 5.

TABLE 1

| Antibody Chain | Sequence |
|---|---|
| SEQ. ID No. 1: | CAACCTANCGGGAAGTAGCCCTTGACCAGGCATCCCAGAG |
| DNA Encoding | TCACAGAGGAACCAGTTGTATCTCCACACCCAGGGGCCAG |
| WVDC-0357 Heavy | TGGATAGACTGATGGGGGTGTTGTTTTGGCTGAGGAGACG |
| Chain Variable | GTGACTGAGGTTCCTTGACCCCAGCAGTCCATAGCGTAGC |
| Region | CACTACCGTAGCCCCCTCTTGCACAGTAATAGACCGCAGA |
| | GTCCTCAGATGTCAGGCTGCTTAGCTGCATGTAGGCTGTG |
| | CTGGAGGATGTGTCTACAGTCAATGTGGCCTTGTCCTTGA |
| | ACTTTTGAATGTAGTGAGTTTCACTATCAGAAGGGTCAAT |
| | ATTACCAATCCATTCAAGGCCTTGTCCAGGCCTCTGCTTC |
| | ACCCAGTGCATCCAGTAGTTGGTGAAGGTGTAGCCAGAAG |
| | CCTTGCAGGACAGCTTCACTGAAGCCCCAGGCTTCACCAG |
| | CTCAGCCCCAGGCTGCTGCAGTTGGACCTGGGAGTGGACA |
| | CCTGTAGCTGCTGCTACCAAAAAGAGGATGATACAGCTCC |
| | ATCCCATGGTGAGATCCTGTGTGCTTAGTAACTGTAGAGA |
| | GAACAGTGATCTTATCCCCCTGTACTCTCCGTTGATGACC |
| | CCCTGCGTTATAATAA |
| SEQ. ID No. 2: | IITQGVINGEYRGIRSLFSLQLLSTQDLTMGWSCIILFL |
| Amino acid | VAAATGVHSQVQLQQPGAELVKPGASVKLSCKASGYTFT |
| sequence encoded | NYWMHWVKQRPGQGLEWIGNIDPSDSETHYIQKFKDKAT |
| by SEQ ID No.: 1 | LTVDTSSSTAYMQLSSLTSEDSAVYYCARGGYGSGYAMD |
| | CWGQGTSVTVSSAKTTPPSVYPLAPGCGDTTGSSVTLGC |
| | LVKGYFPXG |
| SEQ. ID No. 3: | TTTTTAAACACGACTGAGGNCCTCCAGATGTTAACT |
| DNA Encoding | GCTCACTGGATGGTGGGAAGATGGATACAGTTGGTG |
| WVDC-0357 Light | CAGCATCAGCCCGTTTTATTTCCAGCTTGGTCCCCC |
| Chain Variable | ATCCGAACGTATACGGGACATGTGTAACTTGGAGGC |
| Region | AGAAATAAACTCCCAAATCCTCACCCTCCACTCTGC |
| | TAATCTTGAGTGTGAAATCTGTCCCTGAACCACTGC |
| | CACTGAACCTGGCTACGACCCCACAAAATCGGATGG |
| | AAACCCTGTAAATCAGGAGCTGTGGAGACTGGCCTG |
| | GTTTCTGGAGGTACCAGTTCAAATAGGTGTTTCCAT |
| | TACTGTTTTCAAGGCTCTGACTAGACCTGCAAGAGA |
| | TGGAGGCTTGATCTCCAAGACTGACAGGCAGGGAGA |
| | GTGGAGTTTGGGTCATCACAGCATCACTGCTGGAAGC |
| | AGGAATCCAGAACAACAGCACCAACAACCTAACAGGCA |
| | ACTTCATTTTGAGGAGGCAACCTGAGGAGACAGCCT |
| | GAGGAGACTGATCACTCCCCATGTACTCTGCGTTGA |
| | TACCACTGCTTAAAA |
| SEQ. ID No. 4: | LSSGINAEYMGSDQSPQAVSSGCLLKMKLPVRLLVLL |
| Amino acid | FWIPASSSDAVMTQTPLSLPVSLGDQASISCRSSQSL |
| sequence encoded | ENSNGNTYLNWYLQKPGQSPQLLIYRVSIRFCGVVAR |
| by SEQ ID No.: 3 | FSGSGSGTDFTLKISRVEGEDLGVYFCLQVTHVPYTF |
| | GWGTKLEIKRADAAPTVSIFPPSSEQLTSGXPQSCLK |
| SEQ. ID No. 5: | CCACCTATGGGAAGTAGCCCTTGACCAGGCATCCCAGAGT |
| DNA Encoding | CACAGAGGAACCAGTTGTATCTCCACACCCAGGGGCCAGT |
| WVDC-0496 Heavy | GGATAGACTGATGGGGGTGTTGTTTTGGCTGAGGAGACGG |
| Chain Variable | TGACTGAGGTTCCTTGACCCCAGCAGTCCATAGCGTAGCC |
| Region | ACTACCGTAGCCCCCTCTTGCACAGTAATAGACCGCAGAG |
| | TCCTCAGATGTCAGGCTGCTTAGCTGCATGTAGGCTGTGC |
| | TGGAGGATGTGTCTACAGTCAATGTGGCCTTGTCCTTGAA |
| | CTTTTGAATGTAGTGAGTTTCACTATCAGAAGGGTCAATA |
| | TTACCAATCCATTCAAGGCCTTGTCCAGGCCTCTGCTTCA |
| | CCCAGTGCATCCAGTAGTTGGTGAAGGTGTAGCCAGAAGC |
| | CTTGCAGGACAGCTTCACTGAAGCCCCAGGCTTCACCAGC |
| | TCAGCCCCAGGCTGCTGCAGTTGGACCTGGGAGTGGACAC |
| | CTGTAGCTGCTGCTACCAAAAAGAGGATGATACAGCTCCA |
| | TCCCATGGTGAGATCCTGTGTGCTTAGTAACTGTAGAGAG |
| | AACAGTGATCTTATCCCCCTGTACTCTGCGTTGATACCAC |
| | TGCTTATAAAA |
| SEQ. ID No. 6: | FISSGINAEYRGIRSLFSLQLLSTQDLTMGWSCIILFLVA |
| Amino acid | AATGVHSQVQLQQPGAELVKPGASVKLSCKASGYTFTNYW |
| sequence encoded | MHWVKQRPGQGLEWIGNIDPSDSETHYIQKFKDKATLTV |
| by SEQ ID No.: 5 | DTSSSTAYMQLSSLTSEDSAVYYCARGGYGSGYAMDCWGQ |
| | GTSVTVSSAKTTPPSVYPLAPGCGDTTGSSVTLGCLVKGYFP |
| SEQ. ID No. 7: | TCACTTAAACCCGACTGAGGNCCTCCAGATGTTAACTGCT |
| DNA Encoding | CACTGGATGGTGGGAAGATGGATACAGTTGGTGCAGCATC |
| WVDC-0496 Light | AGCCCGTTTTATTTCCAGCTTGGTCCCCGATCCGAACGTA |
| Chain Variable | TACGGGACATGTGTAACTTGGAGGCAGAAATAAACTCCCA |
| Region | AATCCTCAGCCTCCACTCTGCTAATCTTGAGTGTGAAATC |
| | TGTCCCTGAACCACTGCCACTGAACCTGTCTAGGACCCCA |
| | CAAAATCGGTTGGAAACCCTGTAAATCAGGAGCTGTGGAG |
| | ACTGGCCTGGTTTCTGGAGGTACCAGTTCAAATAGGTGTT |
| | TCCATTACTGTTTTCAAGGCTCTGACTAGACCTGCAAGAG |
| | ATGGAGGCTTGATCTCCAAGACTGACAGGCAGGGAGAGTG |
| | GAGTTTGGGTCATCACAGCATCACTGCTGGAAGCAGGAAT |
| | CCAGAACAACAGCACCAACAACCTAACAGGCAACTTCATT |
| | TTGAGGAGGCAACCTGAGGAGACAGCCTGAGGAGACTGAT |
| | CACTCCCCATGTACTCTGCGTTGATACCACTGATTAAA |
| SEQ. ID No. 8: | LISGINAEYMGSDQSPQAVSSGCLLKMKLPVRLLVLLFWIPASSS |
| Amino acid | DAVMTQTPLSLPVSLGDQASISCRSSQSLENSNGNTYLNWYLQKP |
| sequence encoded | GQSPQLLIYRVSNRFCGVLDRFSGSGSGTDFTLKISRVEAEDLGV |
| by SEQ ID No.: 7 | YFCLQVTHVPYTFGSGTKLEIKRADAAPTVSIFPPSSEQLTSGXP |

TABLE 1a
CDR Sequence Fragments

| | SEQ ID No. | | | |
|---|---|---|---|---|
| CDRs | 2 | 4 | 6 | 8 |
| CDR1 | Residues 79-83 | Residues 69-84 | Residues 78-82 | Residues 69-84 |
| CDR2 | Residues 98-114 | Residues 100-106 | Residues 97-113 | Residues 100-106 |
| CDR3 | Residues 147-157 | Residues 139-147 | Residues 146-156 | Residues 139-147 |

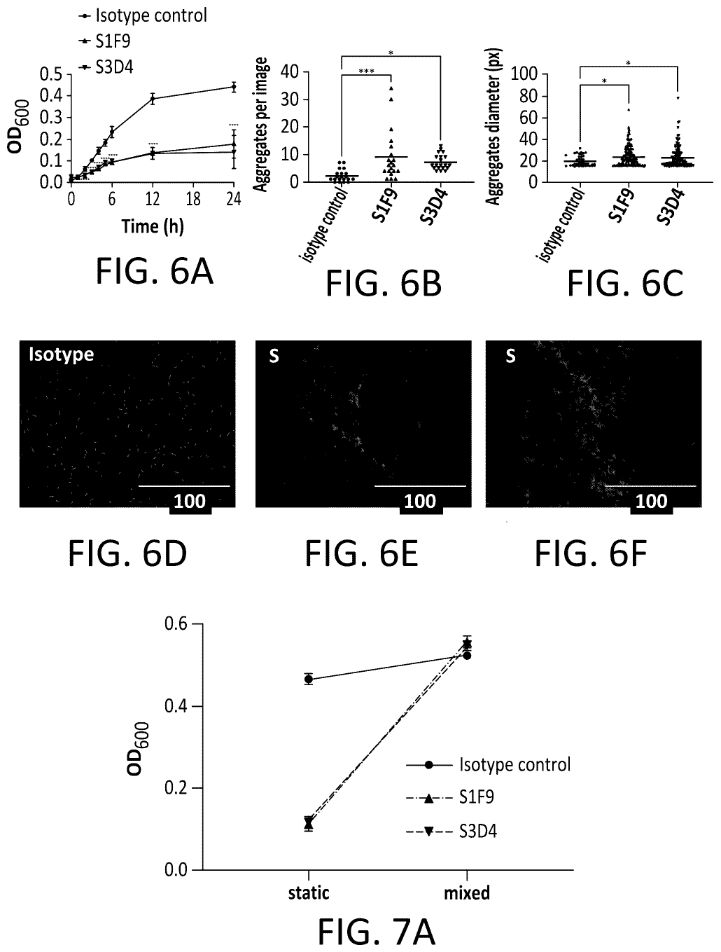

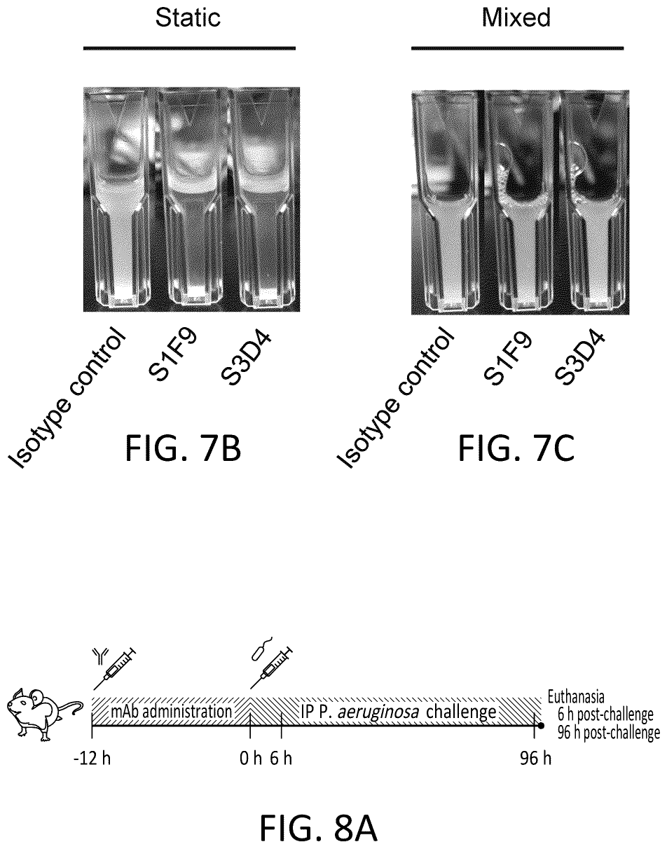

To examine the role of mAbs WVDC-0357 (WVDC-0357 (S1F9)) and WVDC-0496 (S3D4) in inducing agglutination of P. aeruginosa, a bacterial growth assay was performed whereby PAO1 was cultured in lysogeny broth with the inclusion of mAb WVDC-0357 (SIF9), mAb WVDC-0496 (S3D4), or an isotype control mAb. The OD600 was measured over the course of 24 h and absorbance values were significantly decreased with cultures containing mAbs WVDC-0357 (S1F9) and WVDC-0496 (WVDC-0496 (S3D4)) compared to the isotype control mAb, with significant differences (P<0.0001) in absorbance at all time points starting at 2 h, as shown in FIG. 6A. FIG. 7 shows that this difference in absorbance was associated with the presence of bacterial aggregates, which were visually apparent in the growth cultures containing mAbs WVDC-0357 (S1F9) and WVDC-0496 (WVDC-0496 (S3D4)). To further validate the presence of agglutination, PAO1 was stained with BacLight red bacterial stain, treated with mAb WVDC-0357 (S1F9), mAb WVDC-0496 (WVDC-0496 (S3D4)), or an isotype control mAb, and visualized with fluorescence microscopy for bacterial aggregates. Visualization of fluorescent PAO1 treated with mAbs WVDC-0357 (S1F9) and WVDC-0496 (WVDC-0496 (S3D4)) demonstrated an increase in bacterial aggregates compared to the isotype control, as shown in FIGS. 6D to 6F. To quantify the overall representation of bacterial aggregates, randomly imaged areas were analyzed for number and size of bacterial aggregates. For mAbs WVDC-0357 (S1F9) and WVDC-0496 (S3D4), there was a significant increase in the total number of aggregates per image, as shown in FIG. 6B. Furthermore, FIG. 6C shows that the individual aggregate diameter was significantly greater for mAbs WVDC-0357 (S1F9) and WVDC-0496 (S3D4). All together, these results demonstrate that mAbs WVDC-0357 (SIF9) and WVDC-0496 (S3D4) mediate agglutination of P. aeruginosa.

MAbs WVDC-0357 (S1F9) and WVDC-0496 (S3D4) Protect Mice Against Lethal P. aeruginosa Bloodstream Infection.

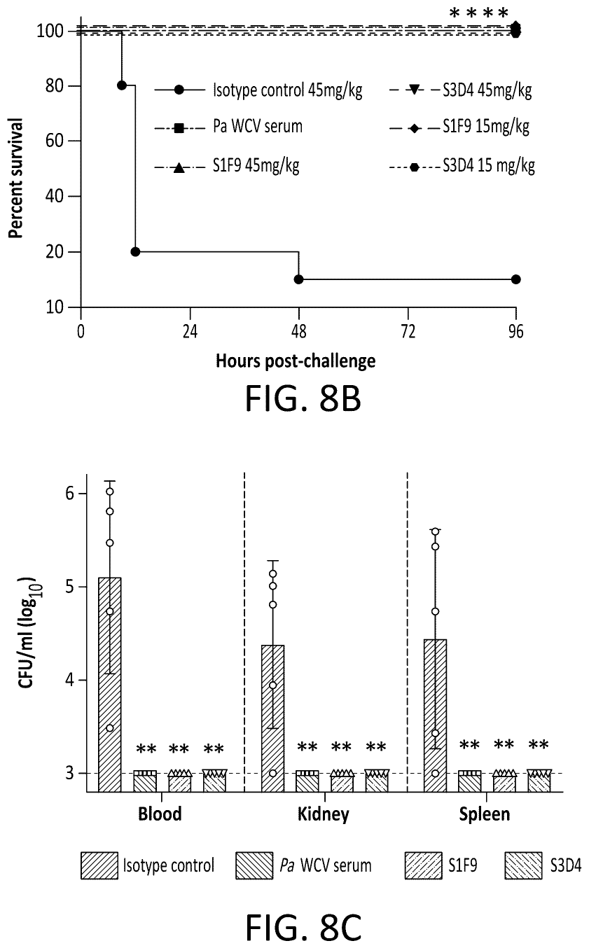

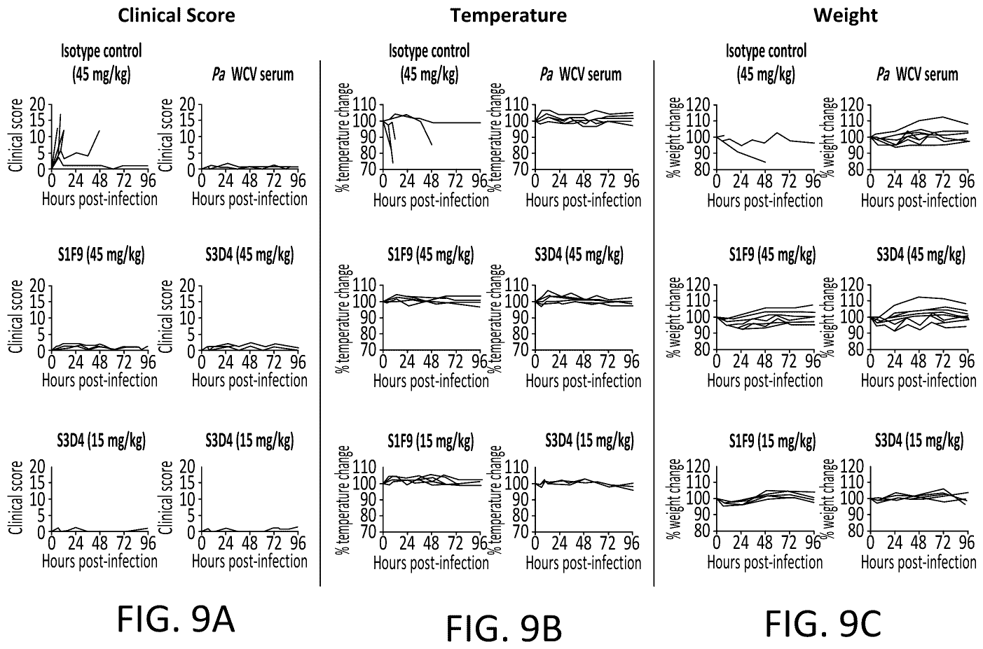

Primary bloodstream infections are one of the most lethal complications of P. aeruginosa infections. Given the functional profile of mAbs WVDC-0357 (SIF9) and WVDC-0496 (S3D4) against P. aeruginosa, the efficacy of these mAbs in a lethal sepsis mouse model of infection was evaluated. In this model, mice were intraperitoneally (IP) administered either an isotype control mAb, P. aeruginosa whole cell vaccinated (Pa WCV) serum, mAb WVDC-0357 (S1F9), or mAb WVDC-0496 (S3D4), challenged 12 h later with a lethal IP dose of PAO1 (5×10⁵ CFU), and monitored for survival over the course of 96 h, as shown in FIG. 8A. A majority of the mice treated with the isotype control mAb had succumbed to infection within the first 24 h, and overall there was only a 10% survival rate at the conclusion of the observation period, as shown in FIG. 8B. In contrast, mice treated with Pa WCV serum, mAb WVDC-0357 (SIF9), or mAb WVDC-0496 (S3D4) were fully protected and had a 100% survival rate. This study was repeated with the addition of a 15 mg/kg concentration group for mAbs WVDC-0357 (SIF9) and WVDC-0496 (S3D4), which still demonstrated a 100% survival rate. To assess the severity of disease, mice were scored daily for clinical outcomes such as appearance, activity, eye closure, respiration quality, body temperature, and body weight. Each variable was given a score ranging from 0 to 4, and mice that had a total cumulative score of 12 or a score of 4 in any individual category were euthanized. During the 96-hour observation period, mice in the isotype control group quickly showed an increase in clinical scores and most of them succumbed to infection between 6 and 12 hours after infection. As a secondary metric of health, temperature was most closely correlated with the survival outcome of mice. Mice that did not survive had on average a rectal temperature of 31.5° C., which was a 15% decrease from baseline. In contrast, mice from groups Pa WCV serum, mAb WVDC-0357 (SIF9), and mAb WVDC-0496 (S3D4) maintained baseline rectal temperatures throughout the whole observation period, as shown in FIG. 9B.

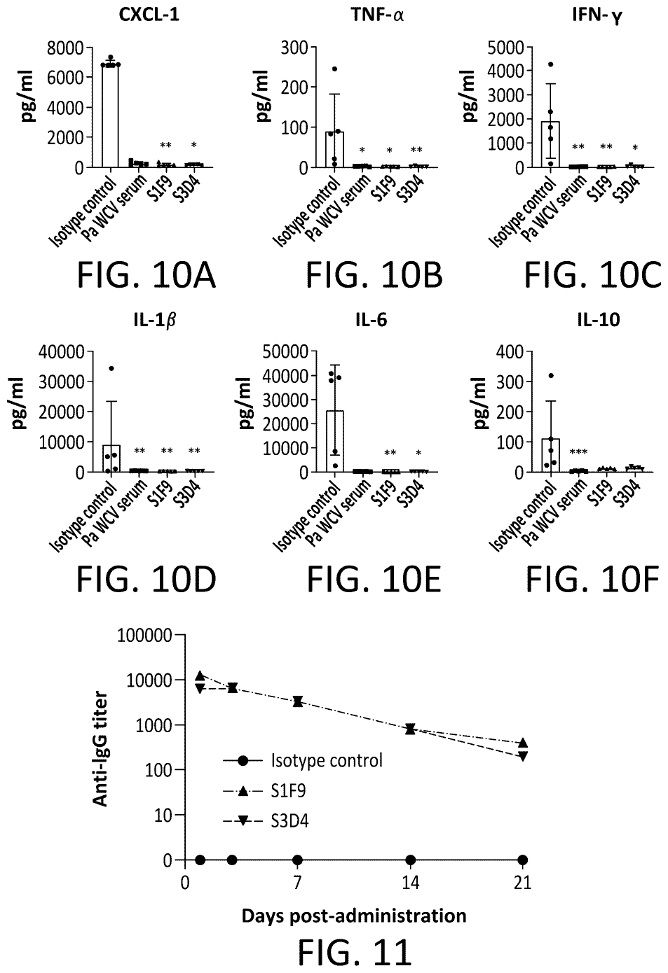

An additional set of mice were euthanized at 6 h post-challenge to quantify the level of bacterial burden and cytokines in the early stages of infection. MAbs WVDC-0357 (S1F9) and WVDC-0496 (S3D4) significantly reduced CFUs to the limit of detection in the blood, kidney, and spleen, as shown in FIG. 8C. This reduction in CFUs was the same as the positive control Pa WCV serum group. Compared to the isotype control mAb group, protected mice had a 2-3 fold log reduction in CFUs. The level of inflammatory cytokines and chemokines was dramatically reduced in mice treated with mAbs WVDC-0357 (SIF9) and WVDC-0496 (S3D4) compared to isotype control mice, as shown in FIG. 10. The concentration of cytokines and chemokines was similar between the positive control Pa WCV serum group and mAbs WVDC-0357 (S1F9) and WVDC-0496 (S3D4), which was near the limit of detection. For mAbs WVDC-0357 (S1F9) and WVDC-0496 (S3D4), there were significant reductions in the levels of CXCL-1, IFN-γ, TNF-α, IL-βB and IL-6.

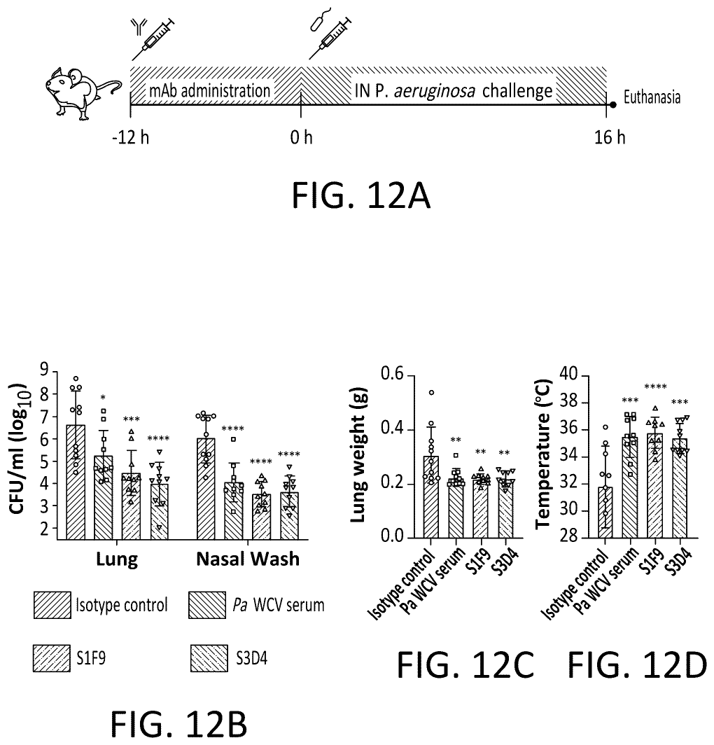

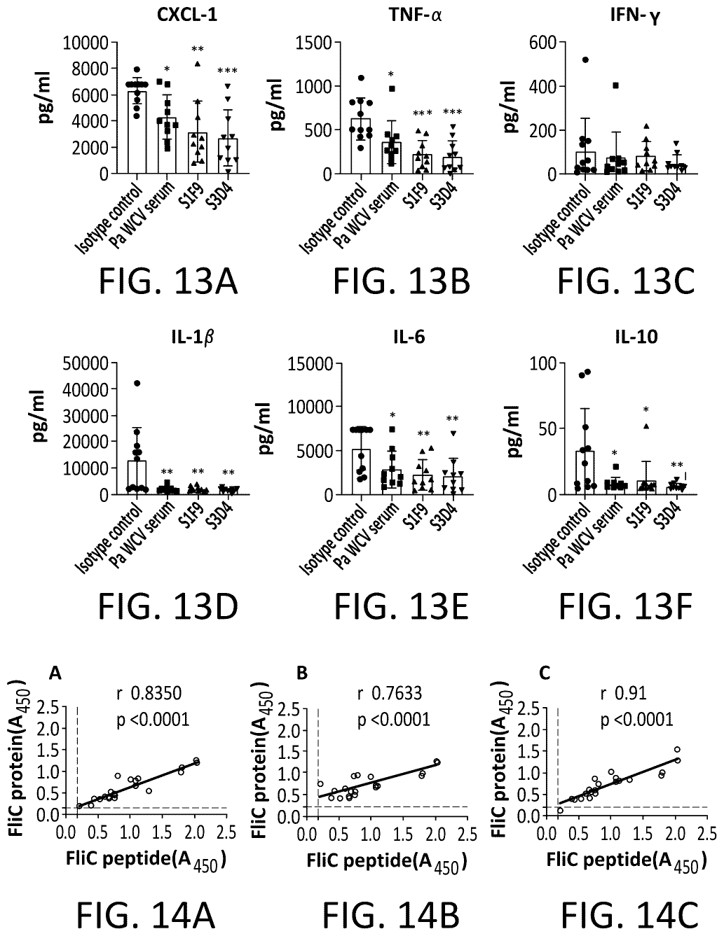

FIG. 12 shows pharmacokinetics of mAbs WVDC-0357 (S1F9) and WVDC-0496 (S3D4) in mice. Six-week old, female CD-1 mice were administered an isotype control mAb, mAb WVDC-0357 (S1F9), or mAb WVDC-0496 (S3D4) at 45 mg/kg. Mice were submandibular bled over the course of 21 days to assess serum IgG titers. As shown in FIG. 13, female CD-1 mice administered an isotype control mAb had little or no serum IgG at any time. However, female CD-1 mice administered mAb WVDC-0357 (S1F9) or mAb WVDC-0496 (S3D4) had a serum IgG titer of 100 to 1000 21 days after mAb administration.

MAbs WVDC-0357 (S1F9) and WVDC-0496 (S3D4) Protect Mice Against Acute P. aeruginosa Lung Infection.

The efficacy of mAbs WVDC-0357 (S1F9) and WVDC-0496 (S3D4) was evaluated in an acute aspiration pneumonia model, which is one of the most common clinical presentations of P. aeruginosa. In this model, mice were IP administered either an isotype control mAb, Pa WCV serum, mAb WVDC-0357 (S1F9), or mAb WVDC-0496 (S3D4), challenged 12 h later with an intranasal dose of PAO1 (2×10⁷ CFU), and euthanized at 16 h post-challenge to look at correlates of protection (FIG. 12A). There were significant reductions in bacterial burden in the respiratory tract of mice treated with mAbs WVDC-0357 (SIF9) and WVDC-0496 (S3D4). When compared to isotype control treated mice, there was a 2.16 log reduction in lung CFUs (P<0.001) and 2.46 log reduction in nasal wash CFUs (P<0.0001) for mAb WVDC-0357 (SIF9) treated mice, and a 2.71 log reduction in lung CFUs (P<0.0001) and 2.35 log reduction in nasal wash CFUs (P<0.0001) for mAb WVDC-0496 (S3D4) treated mice (FIG. 12B). This reduction in bacterial burden was similar to mice treated with Pa WCV serum, which acted as the positive control. Lung weight was used as a marker of lung edema, which revealed that mice treated with the isotype control mAb had a significant increase in lung weight (FIG. 12C). As an overall measure of health status, the rectal temperature of mice was recorded. The average rectal temperature of mice in the Pa WCV serum, mAb WVDC-0357 (S1F9), and mAb WVDC-0496 (S3D4) groups were maintained close to physiological temperature with only a 2-3% decrease (FIG. 12D). In contrast mice from the isotype control group, had an average decrease in rectal temperature by 13.2% (FIG. 12D). Lung cytokine and chemokine levels were determined in order to to assess the presence of acute inflammation in the lung. The lungs from mice treated with mAbs WVDC-0357 (S1F9) and WVDC-0496 (S3D4) had significantly lower levels of inflammatory cytokines and chemokines (CXCL-1, TNF-α, IL-βB and IL-6, and IL-10) when compared to the isotype control, as shown in FIG. 13. Taken together, mAbs WVDC-0357 (SIF9) and WVDC-0496 (S3D4) are able to protect mice from infection and limit the effects hyperinflammation.

P. aeruginosa is one of the most commonly encountered nosocomial pathogens and responsible for complications such as ventilator-associated pneumonias and primary bloodstream infections. Considering the prevalence of MDR strains of P. aeruginosa and mortality associated with P. aeruginosa infections, there is an urgent need to develop alternative therapeutics. In this study, WVDC-0357 (S1F9) and WVDC-0496 (S3D4), which are mAbs that are highly specific to the polysaccharide chain of P. aeruginosa from serogroups 05, were generated. Prophylactic administration of mAbs WVDC-0357 (SIF9) and WVDC-0496 (S3D4) was highly effective at protecting mice against lethal bacteria and pneumonia.

When PAO1 is grown on PIAAMV, a host of genes are differentially regulated, including an upregulation in genes for LPS synthesis. Furthermore, when mice were infected with PAO1-PIAAMV, there was an attenuation in virulence and improvement in overall survival. The predominant antibody response was primarily against LPS. Convalescent mice who had been infected with PAO1-PIAAMV were used to raise therapeutic antibodies against P. aeruginosa. B cells from PAO1-PIAAMV convalescent mice were used for hybridoma generation, and two mAbs, WVDC-0357 (S1F9) and WVDC-0496 (S3D4), were identified that consistently screened positive against intact PAO1. Furthermore, sequencing of the variable regions of WVDC-0357 (S1F9) and WVDC-0496 (S3D4) revealed that these mAbs share the same germline genes, suggesting clonal selection of this particular epitope.

Through ELISA and western blotting, mAbs WVDC-0357 (S1F9) and WVDC-0496 (S3D4) were demonstrated to bind specifically to the outer polysaccharide of P. aeruginosa from serogroup O5 strains. Though the breadth of binding is limited to a subset of strains, mAbs WVDC-0357 (S1F9) and WVDC-0496 (S3D4) were found to be highly effective in preventing the onset of acute P. aeruginosa infection. Despite limitations in specificity with LPS-based therapies, they have been known to mediate high levels of immunity against P. aeruginosa. There are still major challenges to developing broadly protective therapeutics against P. aeruginosa. There are conserved regions of LPS, namely the core and common polysaccharide antigen, however mAbs against these regions have not been shown to mediate strong immunity. Considering the many phenotypes and virulence factors of P. aeruginosa, perhaps the best strategy is to tailor therapy towards specific strains of P. aeruginosa.

P. aeruginosa can be classified into 12 serogroups, based on the sequence and structure of the OSA gene cluster. Using the Pseudomonas database, all genomes with a bloodstream source of infection were identified, and each genome was analyzed for its serogroup using the Pseudomonas aeruginosa serotyper (PAst). Out of 207 genomes with a bloodstream source of infection, 05 was the third most frequent serogroup at 14.5%. Furthermore, the combined frequency of 05/06/011 serogroups was 61.4% of genomes. One potential solution to the challenge of P. aeruginosa therapeutic development is to generate a “cocktail” of mAbs against the most prevalent serogroups. However, there are few reports on anti-LPS mAbs against P. aeruginosa, and most are directed against serogroups 06 and 011.

The current disclosure describes an effective mAb therapy against P. aeruginosa serogroup 05 with efficacy in multiple models of infection. When untreated mice are infected with a lethal dose of P. aeruginosa, they typically succumb to infection within 24 to 48 h, with pathogenicity driven in large part by endotoxic shock. In a mouse model of infection, prophylactic administration of mAbs WVDC-0357 (S1F9) and WVDC-0496 (S3D4) was able to significantly reduce bacterial burden and improve survival of mice. In addition, mAbs WVDC-0357 (SIF9) and WVDC-0496 (S3D4) mediate agglutination, which is a mechanism that can facilitate clearance of the pathogen. Limiting the spread of infection is invariably important in preventing the onset of lethal endotoxic shock. Dysregulation of pro-inflammatory cytokines (TNF-α, IL-1ß and IL-6) and anti-inflammatory cytokines (IL-10) are often associated with life-threatening infection or presence of endotoxin. The chemokine CXCL-1 is associated with neutrophil recruitment and is important in combating early infection, though prolonged neutrophil accumulation is associated with deleterious inflammation. In models directed to sepsis and/or pneumonia, the cytokine and chemokine profile suggests that mAbs WVDC-0357 (S1F9) and WVDC-0496 (S3D4) were able to attenuate severe infection and endotoxic shock from taking place.

The present disclosure describes the generation and characterization of new mAbs directed against P. aeruginosa from serogroup 05. These mAbs are highly protective in sepsis and pneumonia models of P. aeruginosa infection by increasing bacterial clearance and preventing endotoxic shock. Future work involves translating mAbs WVDC-0357 (S1F9) and WVDC-0496 (S3D4) towards clinical applications, which entails humanization of these mAbs and further translational efforts. In addition, mAbs WVDC-0357 (S1F9) and WVDC-0496 (S3D4) only bind to a subset of P. aeruginosa strains, and a collaborative effort should be made to combine existing anti-LPS mAbs into one therapy against the most prevalent serogroups of P. aeruginosa.

Flic Antibodies Against P. aeruginosa and B. Pseudomallei

Various embodiments disclosed herein are directed to methods of generating mAbs which are able to bind to an antigen conserved across different Gram-negative organisms such as P. aeruginosa and B. pseudomallei. In this regard, flagellin or FliC has been considered as a potential candidate for mAb based therapies. FliC is the protein subunit of bacterial flagella that contributes to bacterial motility. It is an abundant protein located on the surface of B. pseudomallei, P. aeruginosa and other ESKAPE pathogens essential for the establishment of the infections. It is highly immunogenic and can elicit an inflammatory response via TLR5 activation. Since flagellin is broadly conserved across Burkholderia species, P. aeruginosa and other Gram-negative organisms, targeting antibody development to the conserved regions of the flagellin protein allows generation of antibodies that would broadly bind the flagellum of different motile pathogenic species.

In this study, a novel mAb named WVDC-2109 was generated using hybridoma-based technology. WVDC-2109 is active against P. aeruginosa and B. pseudomallei FliC. By using a synthetic peptide based strategy of immunization, an mAb able to broadly recognize more than one pathogen was generated. WVDC-2109 contributes to complement-mediated killing of P. aeruginosa as well as opsonophagocytic killing by macrophages. Prophylactic activity in a pre-clinical murine model of acute pneumonia was observed. Prophylactic activity was also observed in a model of sepsis. The present disclosure describes a promising mAb candidate for anti-P. aeruginosa therapy as well as additional therapeutic options for other flagellated pathogens.

Generation of Broadly Reactive mAbs Against P. aeruginosa and B. pseudomallei

Previous studies have demonstrated that mAbs designed against surface components can be protective against infections caused by Burkholderia species as well as P. aeruginosa. In order to generate mAbs able to broadly recognize and bind P. aeruginosa and B. pseudomallei, a peptide covering the conserved regions of flagellin of both pathogens was designed. The peptide was conjugated to the carrier protein diphtheria toxoid CRM 197. Mice were vaccinated with the FliC-CRM peptide and inguinal lymph nodes and splenocytes from the vaccinated mice showing the highest antibody titer against P. aeruginosa and B. pseudomallei were extracted and fused with myeloma cells. Resulting hybridomas were cultured and subjected to several screenings. A direct ELISA assay was used to select for hybridomas producing IgG against FliC peptide, recombinant FliC, P. aeruginosa PAO1 and B. pseudomallei Bp82 above detection threshold. Of the total hybridomas produced, 5.1% were selected after the last screening. For all hybridomas selected, binding to FliC peptide correlated with binding to recombinant protein, B. pseudomallei Bp82 and P. aeruginosa PAO1, as shown in FIGS. 14A to 14C. Among these hybridomas, a hybridoma producing an IgG2bK antibody, called WVDC-2109, was selected. This antibody was purified and subjected to further characterization studies.

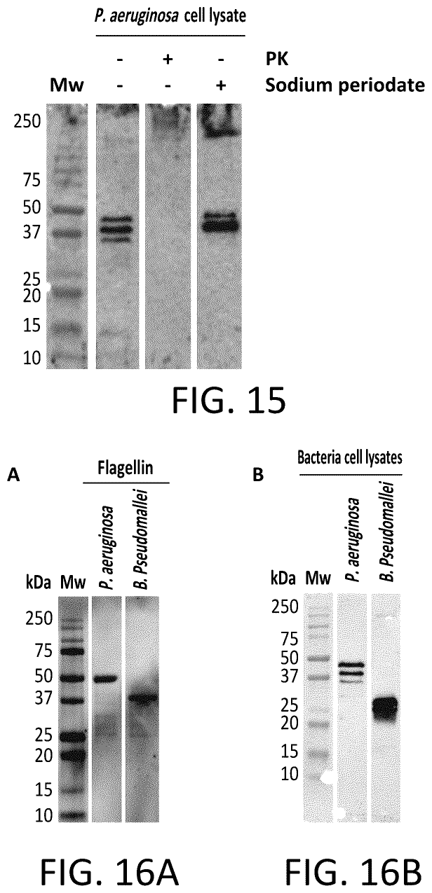

The next step was to confirm WVDC-2109 antigen. Treatment of P. aeruginosa PAO1 lysates with proteinase K abolished antigen recognition by WVDC-2109 while treatment with sodium periodate did not, as shown in FIG. 15. This suggests that the antigen is not a polysaccharide, but it is a protein. By Western blotting and using purified recombinant FliC from P. aeruginosa and B. pseudomallei, WVDC-2109 was determined to bind to flagellin from both pathogens, as shown in FIG. 16A. Furthermore, Western blot analysis also confirmed the ability of the mAb to recognize whole cell lysates from both targeted pathogens, P. aeruginosa and B. pseudomallei, as shown in FIG. 16B. Altogether, these data support that WVDC-2109 recognizes the FliC protein at the surface of P. aeruginosa PAO1 and B. pseudomallei Bp82.

Referring to Table 3, the variable chain sequences of WVDC-2109 are presented. The heavy chain antibody DNA sequence of WVDC-2109 has SEQ. ID No.: 9. The heavy chain antibody protein sequence of WVDC-2109 has SEQ. ID No.: 10. The light chain antibody DNA sequence of WVDC-2109 has SEQ. ID No.: 11. The light chain antibody protein sequence of WVDC-2109 has SEQ. ID No.: 12.

In Table 3, the CDRs in SEQ ID Nos.: 10 and 12 are shown in bold underlined text, while framework regions are shown in bold italic text. Table 3a identifies the portions of SEQ ID Nos.: 10 and 12 corresponding to the various CDRs. The CDRs determine specific antibody binding to, in this case, a flagellin protein.

WVDC-2109 Interacts with Flagellin without Blocking TLR5 Activation.

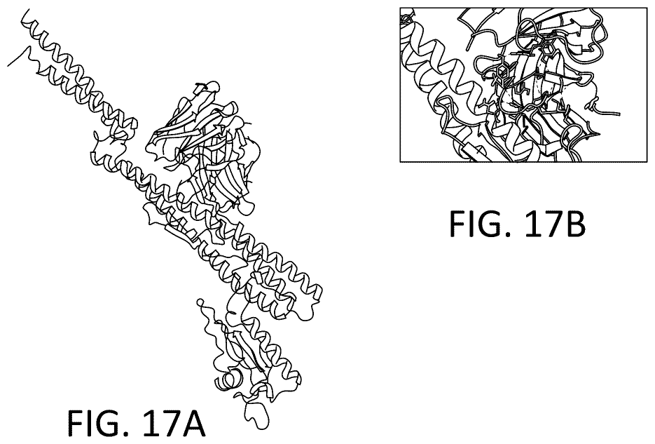

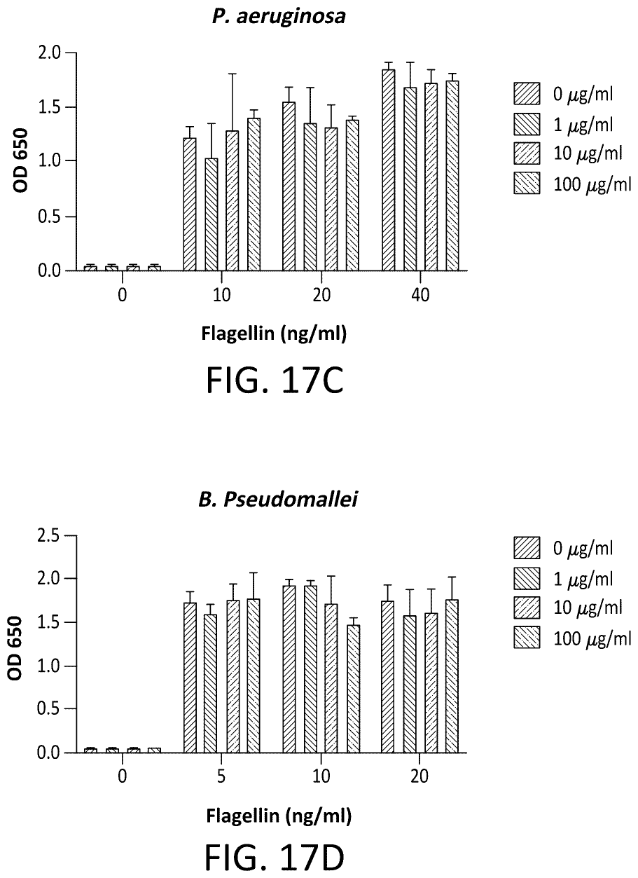

It has been previously demonstrated that, unlike other Toll like receptors (TLRs) such as TLR3 and TLR9, TLR5-mediated immune stimulation does not increase the risk of exacerbated inflammation and tissue destruction. On the contrary, host inflammatory response via TLR5 seems to be protective in P. aeruginosa infections. Using computational tools, the residues involved in the WVDC-2109-flagellin interaction were predicted, as shown in FIGS. 17A and 17B. Residues 51-90 in the FliC domain 1 are recognized by WVDC-2109 . . . . Some of those residues also mediate FliC-TLR5 interaction, although more amino acids are involved in this interaction. To investigate if the presence of WVDC-2109 antibody could interfere on TLR5 activation, purified recombinant FliC from P. aeruginosa and B. pseudomallei was incubated with the mAb. After 90 min of incubation, the mixture was used to activate human HEK-Blue™-hTLR5 cells. Flagellin from both pathogens was immunostimulatory, even using low concentrations of protein. The presence of the antibody did not block the activation of the cells mediated by FliC, at least at the concentrations used, as shown in FIGS. 17C and 17D.

TABLE 3

| Antibody Chain | Sequence |
|---|---|
| SEQ. ID No. 9: | GAGGTGCAGCTGCAGGAGTCTGGGGCTGAGCTGGTAAAGCCTGG |
| DNA encoding | GGCTTCAGTGAAGTTGTCCTGTAAGGCTTCTGGCTACACTTTTAC |
| heavy chain | CAGTTACTGGATGCACTGGGTGAAGCAGAGGCCTGGACAAGGCC |
| variable region | TTGAGTGGATTGGAATGATTCGTCCTAATAGTGGTAGTACAAATT |
| at N-terminus of | ATAATGAGAAGTTCAAGAGTAAGGCCACACTGACTGTAGACAAA |
| mature peptide. | TCCTCCAGCACAGCCTACATGCAACTCAGCAGCCCGACATCTGA |
| | GGACTCTGCGGTCTATTACTGTGCAAGAAGGGGCTACGGTAGTA |
| | GTTATGGATACTATGCTATGGACTACTGGGGTCAAGGAACCTCA |
| | GTCACCGTCTCCAGCGCTAAAACAACACCCCCAT |
| | CAGTCTATCCACTGGCCCCTGGGTGTGGAGATACAACTGGTTCC |
| | TCTGTGACTCTGGGATGCCTGGTCAAGGGCTACTTCCCTGAGT |
| SEQ. ID No. 10: | EVQLQESGAELVKPGASVKLSCKASGYTFTSYWMHWVKQRPGQGL |
| Peptide sequence | EWIGMIRPNSGSTNYNEKFKSKATLTVDKSSSTAYMQLSSPTSEDS |
| encoded by SEQ | AVYYCARRGYGSSYGYYAMDYWGQGTSVTVSSAKTTPPSVYPLAP |
| ID No.: 9 | GCGDTTGSSVTLGCLVKGYFPE |
| SEQ. ID No. 11: | GAATTCGCCCTTACGAATTCTGACATTGTGGTGACACAGACTACA |
| DNA encoding | TCCTCCCTGTCTGCCTCTCTGGGAGACAGAGTCACCATCAGTTGC |
| light chain | AGGGCAAGTCAGGACATTAGCAATTATCTAAACTGGTATCAGCA |
| variable region | GAAACCAGATGGAACTGTTAAACTCCTGATCAACTACACATCAA |
| at N-terminus of | GATTAAACTCAGGAGTCCCATCAAGGTTCAGTGGCAGTGGGTCT |
| mature peptide. | GGAACAGATTATTCTCTCACCATTAGCAACCTGGAGCAGGAAGA |
| | TATTGCCACTTACTTTTGCCAACAGGGTAATACGCTTCCGTGGAC |
| | GTTCGGTGGTGGCACCAAGCTCGAGATAAGGGCGAATTC |
| SEQ. ID No. 12: | DIVVTQTTSSLSASLGDRVTISCRASQDISNYLNWYQQKPDGTVKLL |
| Peptide sequence | INYTSRLNSGVPSRFSGSGSGTDYSLTISNLEQEDIATYFCQQGNTL |
| encoded by SEQ | PWTFGGGTKLE |
| ID No.: 11 | |

TABLE 3a
CDR Sequence Fragments

SEQ ID No.

| CDRs | 10 | 12 |
|---|---|---|
| CDR1 | Residues 31-35 | Residues 24-34 |
| CDR2 | Residues 50-66 | Residues 50-56 |
| CDR3 | Residues 99-112 | Residues 89-97 |

WVDC-2109 Increases Complement-Mediated Killing and Opsonophagocytic Killing of P. aeruginosa.

Monoclonal antibodies can induce bacterial clearance via activating complement-mediated lysis or opsonophagocytosis mediated by macrophages. Given the importance of Complement system, many pathogens have developed strategies to evade it, including P. aeruginosa and B. pseudomallei. To determine whether the presence of WVDC-2109 antibody could increase P. aeruginosa PAO1 complement-dependent killing, bacteria was incubated with complement alone or WVDC-2109 and complement. A heat-inactivated complement system was used as a control. Complement alone was not sufficient to significatively decrease bacterial survival. However, the addition of the mAb WVDC-2109 to the complement source decreased bacterial viability by 71.09%.

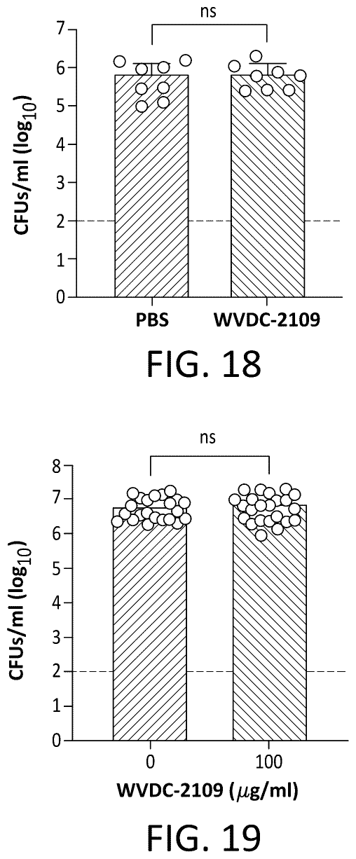

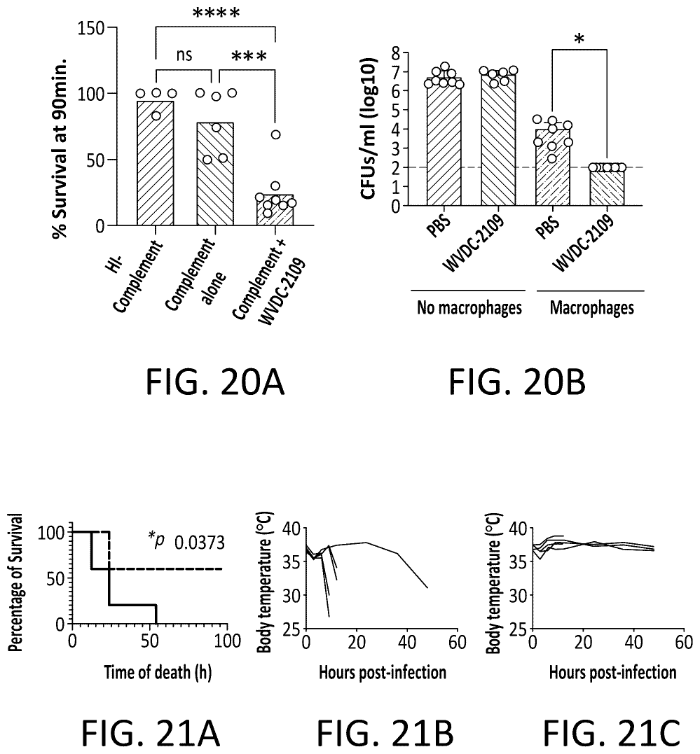

To evaluate if WVDC-2109 could also facilitate bacterial uptake by phagocytic cells, the ability of J774A. 1 macrophages to kill P. aeruginosa PAO1 was examined. PA01 was previously opsonized with the mAb WVDC-2109, or was previously treated with PBS as a control. WVDC-2109 did not increase the attachment of the bacteria to the macrophages, as shown in FIG. 18. Moreover, the antibody alone was not sufficient to reduce bacterial viability, as shown in FIG. 19. However, although bacterial survival was reduced by the presence of macrophages alone, the opsonization of P. aeruginosa with WVDC-2109 led to the killing of an additional 99.06% of bacteria compared to macrophages alone, as shown in FIG. 20B. Overall, these results show that WVDC-2109 can mediate bacterial killing by complement system and macrophages.

WVDC-2109 Protects Against P. aeruginosa Lethal Bacteremia.

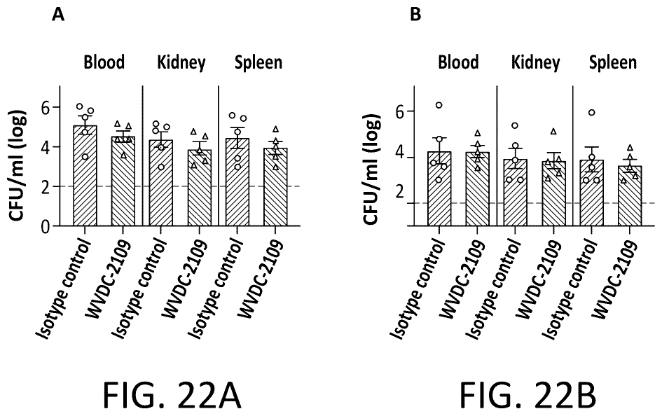

Bloodstream infections (BSI) caused by P. aeruginosa represent 8.5% of all BSIs, with the highest mortality rate among causative agents. To investigate the impact of the WVDC-2109 in P. aeruginosa bloodstream infections, the capacity of the antibody to improve survival was tested. Mice received WVDC-2109 or a non-specific IgG as a control and six hours later they were challenged intraperitoneally with a lethal dose of P. aeruginosa PAO1. Survival as well as morbidity were checked for the subsequent days. While all control mice that received a passive immunization with the non-specific isotype IgG died from infection within the first 60 h after challenge and displayed a dramatic temperature loss (FIG. 22B), WVDC-2109 prophylactic treatment significatively increased survival of mice by 60%, as shown in FIG. 21A.

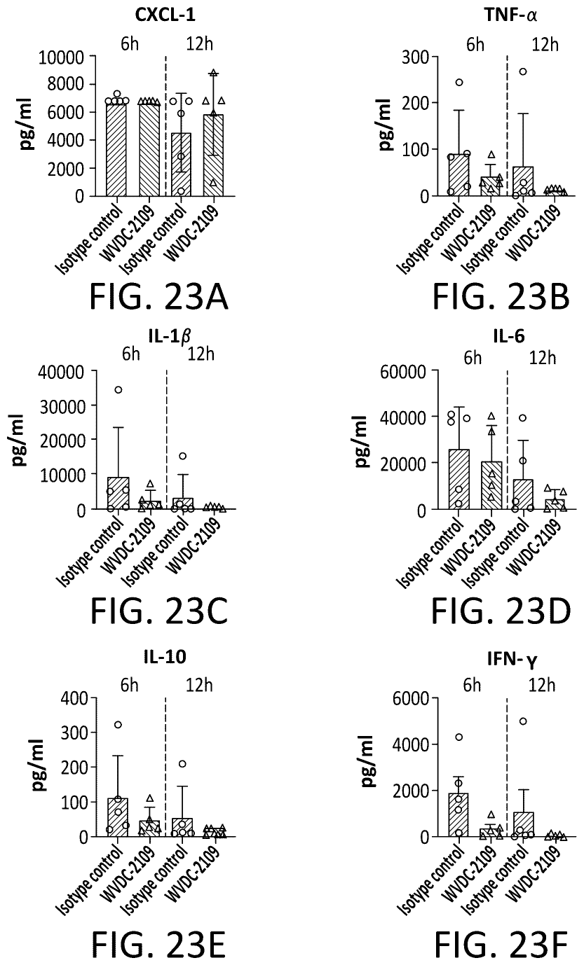

Moreover, although non statistically significant, bacterial burden in blood, kidney and spleen was lower in mice treated with WVDC-2109 compared with mice passively immunized with a non-specific IgG at 6 and 12 h after infection. Cytokine levels (TNFα, IL-βB, IL-6, IL-10, IFNγ) were also lower in the serum of mice that received WVDC-2109, as shown in FIGS. 23A to 23F, indicating less inflammation in those mice.

Overall, these data show that prophylaxis with WVDC-2109 protects against P. aeruginosa bloodstream infection.

WVDC-2109 Reduces Bacterial Burden and Edema in the Respiratory Tract During Murine P. aeruginosa Acute Pneumonia.

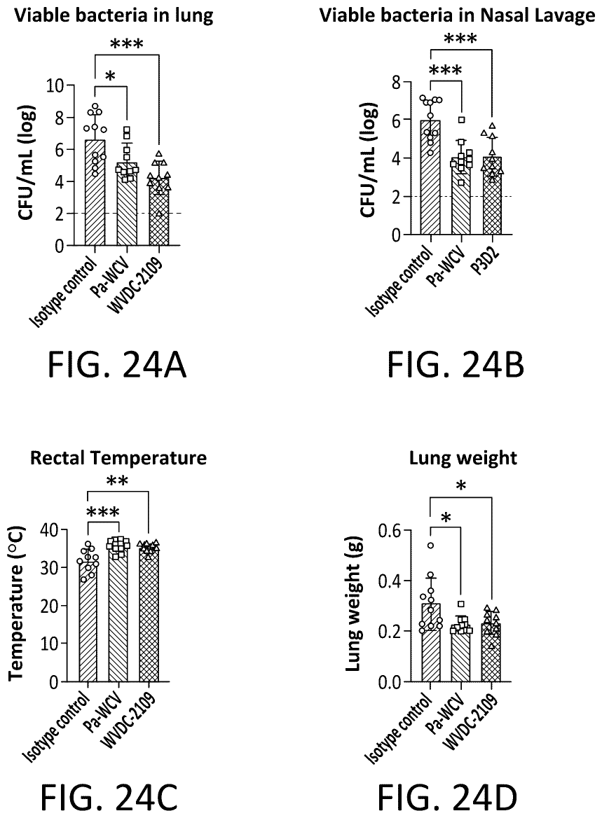

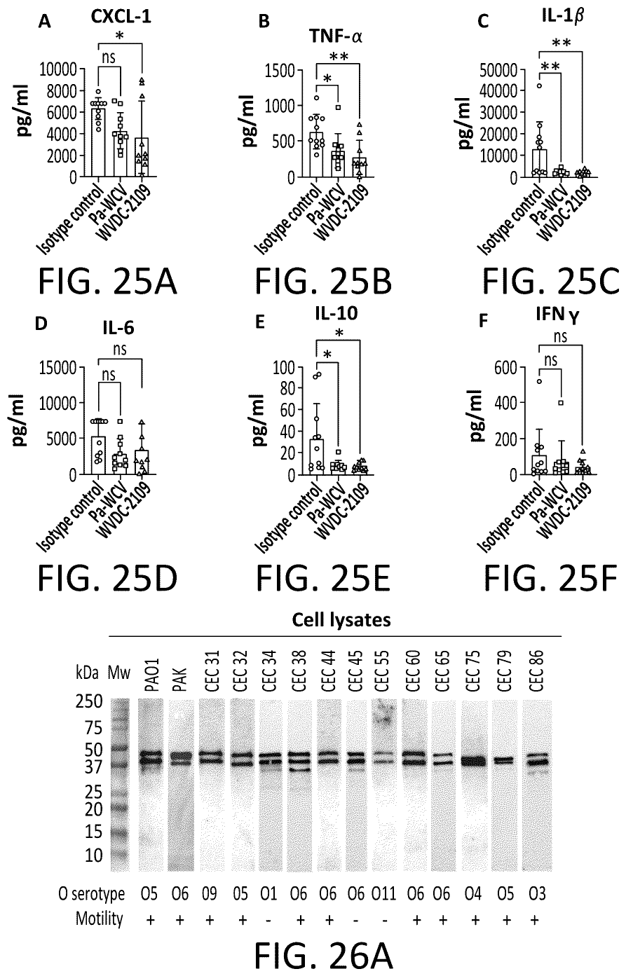

We further evaluated the efficacy of WVDC-2109 in another clinically relevant in vivo model of P. aeruginosa infection: a murine model of acute pneumonia. A passive immunization in outbred CD-1 mice was performed with WVDC-2109 or a non-specific isotype control. As a positive control for protection, mice were immunized with serum from mice previously vaccinated with Pa-WCV. Six hours after injection, mice were challenged intranasally with P. aeruginosa PAO1. Sixteen hours post-challenge, mice were euthanized and rectal temperature, wet lung weight and bacterial burden in the respiratory tract were evaluated. While bacterial loads in the lung and the nares of mice immunized with the isotype control were high, treatment with WVDC-2109 significatively reduced bacterial burden in the lung and in the nares (FIGS. 24A and 24B). WVDC-2109 treatment also increased significatively body temperature and reduced lung wet weight in CD-1 mice, which suggests reduced pulmonary edema in those animals (FIGS. 24C and 24D). To further determine the effect of WVDC-2109 on pulmonary inflammation, cytokines from lung homogenate supernatant were determined. Levels of CXCL-1, TNFα, IL-βB and IL-10 were significatively lower in the lung from mice previously immunized with WVDC-2109 compared with mice that received a non-specific antibody (FIGS. 25A to 25F).

Overall, these data show that prophylaxis with WVDC-2109 can reduce bacterial colonization, edema and inflammation in the lower respiratory tract during P. aeruginosa acute murine pneumonia.

WVDC-2109 Binds to P. aeruginosa Clinical Isolates, Burkholderia Species and Other Pathogens of Concern

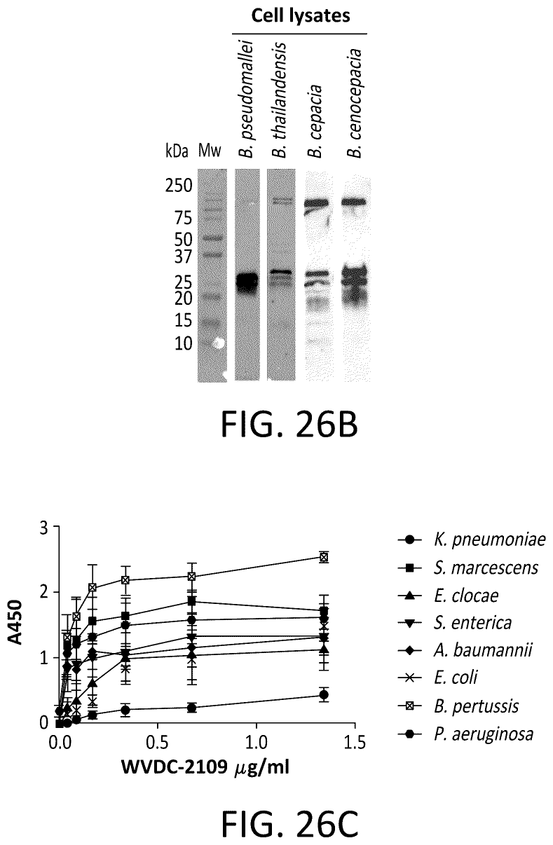

The main goal of this study was to produce a mAb able to cross-react and bind to different pathogens of interest. P. aeruginosa expresses two different types of flagellin, type a flagellin and type b flagellin, with different molecular sizes. Each P. aeruginosa strain produces a single type of flagellin, and no switching between serotypes has been reported for any P. aeruginosa strain. A recent study has reported an association between the O-serotype and flagellin type. In order to determine whether WVDC-2109 was capable of binding different clinical isolates expressing different LPS serotypes, a diverse range of P. aeruginosa strains isolated from CF patients was tested, shown in Table 2 above. Using Western blot, WVDC-2109 was found to be able to bind to clinical isolates from all LPS serotypes tested and to both non-motile and motile clinical strains, as shown in FIG. 26A.

WVDC-2109 was designed by targeting the conserved regions of flagellin of P. aeruginosa and B. pseudomallei. Given that flagellin is broadly conserved across Burkholderia species, the ability of WVDC-2109 was able to bind not only B. pseudomallei Bp82 but other Burkholderia species of interest was tested. WVDC-2109 was able to recognize by Western Blot, not only B. pseudomallei Bp82 but also B. thailandensis, B. cepacia and B. cenocepacia, as seen in FIG. 26B.

Finally, the ability of WVDC-2109 to bind to other pathogens of concern was analyzed by ELISA, as seen in FIG. 26C. WVDC-2109 bound to most of the pathogens tested, especially to Bordetella pertussis (B. pertussis flagellin has 47.21% identity with P. aeruginosa type b flagellin and 38.71% with B. pseudomallei flagellin) and Serratia marcescens (S. marcescens flagellin has 47.2% identity with P. aeruginosa type b flagellin and 41.6% with B. pseudomallei flagellin). It also recognized Salmonella enterica flagellin (38.2% identity with P. aeruginosa type b flagellin and 39.1% identity with B. pseudomallei flagellin), Acinetobacter baumannii flagellin (68.12% identity with P. aeruginosa type b flagellin and 41.28% identity with B. pseudomallei flagellin), Enterobacter clocae flagellin (38.36% identity with P. aeruginosa type b flagellin and 37.81% identity with B. pseudomallei flagellin) and Escherichia coli flagellin (38.82% identity with P. aeruginosa type b flagellin and 37.67% identity with B. pseudomallei flagellin). WVDC-2109 barely bound to the non-flagellated Klebsiella pneumoniae.

### Bispecific Antibodies

A bispecific monoclonal antibody (BsAb) can simultaneously bind to two different antigens or two different epitopes on the same antigen. There are several platforms for construction of a bispecific antibodies or antibody fragments by techniques known in the art.

Hybridoma cells from a first species expressing a first mAb targeting a first antigen or a first epitope of a target antigen may be produced. Hybridoma cells from a second species expressing a second mAb may be produced, where the second mAb targets a second antigen or a second epitope of the target antigen. Hybridoma cells from the first species are hybridized with hybridoma cells from the second species, yielding quadroma cells, which produce bispecific hybrid antibodies. The first species is typically a mouse, and the second species may be a rat.

The hybrid antibodies include:
a first binding site including a heavy chain from the first species paired with a light chain from the first species; and
a second binding site including a heavy chain from the second species paired with a light chain from the second species.

Thus, the hybrid antibodies have a first binding site targeting the first antigen or the first epitope of the target antigen; and a second binding site targeting the second antigen or the second epitope of the target antigen. Heavy chains from one species do not typically bind light chains from a different species, so mismatched hybrid antibodies do not form in substantial quantities.

Thus, for example, hybridoma cells from a mouse expressing WVDC-2109 targeting the flagellin antigen from P. aeruginosa may be hybridized with rat cells expressing a second antibody. The second antibody is not limited to any specific antibody. As an example, the second antibody may be WVDC-0357 (SIF9), targeting a lipopolysaccharide from O5 strains of P. aeruginosa. The resulting quadroma cells will produce bispecific hybrid antibodies targeting both flagellin from multiple P. aeruginosa strains, and a lipopolysaccharide from the O5 strains of P. aeruginosa.

Similarly, hybridoma cells from a mouse expressing WVDC-0357 (SIF9), targeting a lipopolysaccharide from O5 strains of P. aeruginosa, may be hybridized with rat cells expressing a second antibody. The second antibody is not limited to any specific antibody. As an example, the second antibody may be an antibody against lipopolysaccharide from O11 strains of P. aeruginosa, e.g., Panubacumab. The resulting quadroma cells will produce bispecific hybrid antibodies targeting lipopolysaccharides from both the 05 and O11 strains of P. aeruginosa.

Formation of mismatched hybrid antibodies may be reduced further by inducing a mutation in heavy chain constant regions of the antibodies. The “knobs-into-holes” approach replaces a small amino acid in the CH3 domain of one antibody with a large amino acid, generating a knob in the CH3 region. A large amino acid in the CH3 region of the other antibody is replaced with a small amino acid, generating a hole. Two CH3 regions with knobs do not fit together well; similarly, two CH3 regions with holes do not fit together well. Thus, hybrid antibodies with a hole in one CH3 region and a corresponding knob in the other CH3 region are favored.

Other strategies known in the art for preparing bispecific antibodies may be used. These strategies may result in immunoglobulin-like (IgG-like) or non-IgG-like bispecific antibodies. Such IgG-like strategies include the strand-exchange engineered domain (SEED) platform, formation of dual variable domain immunoglobulins (DVD-Ig), and formation of Fabs-in-tandem immunoglobulins (FIT-Ig).

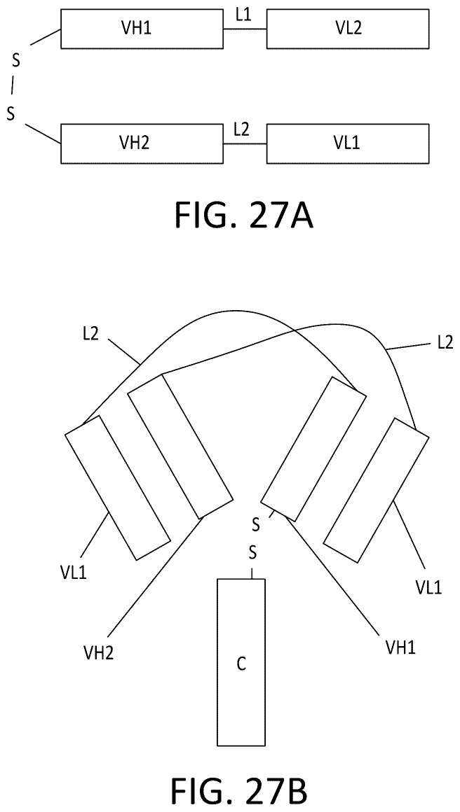

Another strategy involves construction of an antibody fragment such as a dual-affinity re-targeting protein (DART), which involves construction of two variable domain fragments, FV₁ and FV₂. Fv1 consists of a heavy chain from the active site of a first antibody (VH1), linked via a peptide linker L1 to a light chain from a second antibody (VL2), as seen in FIG. 27A. FV₂ consists of a heavy chain from the active site of the second antibody (VH2), linked via a second peptide linker L2 to a light chain from the first antibody (VL1). The heavy chains VH1 and VH2 are linked by a disulfide bond between the C-terminal ends of FV₁ and FV₂. The linked variable domain fragments mimic an antibody binding site, due to the disulfide bond and association between the two FV₁ and FV₂ chains. Alternatively, the disulfide bond may be used to link one of the heavy chains of the bispecific antibody fragment to an immunoglobulin constant domain C or other carrier protein, rather than link the heavy chains to each other, as shown in FIG. 27B.

Referring to Table 1 above, mAb WVDC-0357 (SIF9) and mAb WVDC-0496 (S3D4) each bind a lipopolysaccharide of P. aeruginosa. However, it is probable that they bind to different lipopolysaccharide epitopes. In order to construct an antibody which binds to both lipopolysaccharide epitopes, the heavy chain antibody protein sequence of mAb WVDC-0357 (S1F9) (SEG ID No. 2) may be linked to the light chain antibody protein sequence of mAb WVDC-0496 (S3D4) (SEQ ID No.: 8) through a peptide linker. Similarly, the heavy chain antibody protein sequence of mAb WVDC-0496 (S3D4) (SEG ID No. 6) may be linked to the light chain antibody protein sequence of mAb WVDC-0357 (SIF9) (SEQ ID No.: 4) through a peptide linker. The C-terminal ends of SEQ ID Nos. 2 and 6 may be linked through a disulfide bond, and the chains may be allowed to associate into a bispecific antibody fragment.

In various embodiments, bispecific antibodies which bind to different antigens may be constructed. For example, the antibody mAb WVDC-0357 (S1F9) binds a lipopolysaccharide of P. aeruginosa. The antibody WVDC-2109 binds to the P. aeruginosa flagellin protein (FliC). In order to construct an antibody which binds to both the P. aeruginosa lipopolysaccharide antigen and the P. aeruginosa flagellin antigen, the heavy chain antibody protein sequence of mAb WVDC-0357 (S1F9) (SEG ID No. 2) may be linked to the light chain antibody protein sequence of WVDC-2109 (SEQ ID No.: 12) through a peptide linker. Similarly, the heavy chain antibody protein sequence of WVDC-2109 (SEQ ID No.: 10) may be linked to the light chain antibody protein sequence of mAb WVDC-0357 (S1F9) (SEQ ID No.: 4) through a second peptide linker. The C-terminal ends of SEQ ID Nos. 2 and 6 may be linked through a disulfide bond, and the chains may be allowed to associate into a bispecific antibody fragment. A similar construct may be prepared using heavy and light chain sequences from mAb WVDC-0496 (S3D4).

### Chimeric Antibodies, Humanized Antibodies, and Antibody Fragments

In various embodiments, the heavy and light chains from the monoclonal antibodies WVDC-2109, WVDC-0496 (S3D4), and WVDC-0357 (S1F9) may be used to prepare antibodies configured to be administered to a human. This may be done by preparing chimeric or humanized antibodies.

The DNA sequences coding a variable light chain from a mouse (VL_{M}) may be linked to DNA sequences encoding a human light chain constant region (CL_{H}), where CL_{H} comes from a human immunoglobulin IgG antibody. The DNA sequences coding a variable heavy chain from a mouse (VHM) may be linked to DNA sequences encoding a human heavy chain constant region (CH_{H}), where CH_{H} comes from the human IgG antibody. The resulting mouse/human chimeric genes are transfected into mammalian cells, and expressed as chimeric antibodies.

In various embodiments, the human heavy chain constant region CH_{H} may contain three domains and a hinge region, where:
a first domain CH1 is linked to the variable chain VH_{M};
the hinge region links CH1 to a second domain CH2; and
the second domain CH2 is linked to a third domain CH3.
   The CH1 domain and the light chain constant region CL_{H} may be linked by a disulfide bond. The hinge regions on two human heavy chain constant regions CH_{H} may be linked by disulfide bonds, causing the antibody to form a dimeric structure with two antigen binding sites, each formed by the VL_{M} and VH_{M} regions of the chimeric antibody.

In various embodiments, a chimeric antibody fragment may be constructed by:
linking a DNA sequence coding a mouse variable light chain VL_{M} to a DNA sequence encoding a human light chain constant region CL_{H};
linking a DNA sequence coding a mouse variable heavy chain VH_{M} to a DNA sequence encoding the CH1 region of the human heavy chain constant region CH_{H}; and
transfecting the resulting mouse/human chimeric genes into mammalian cells, expressed them to produce a chimeric antibody fragment Fab lacking the CH2 and CH3 regions.
   In various embodiments, the DNA sequence encoding the CH1 region may also encode the hinge region. Expression of the resulting gene may result in a chimeric antibody fragment dimer F(ab)₂, due to disulfide bonding between the hinge regions of two chimeric Fab fragments. In various embodiments, the DNA sequence encoding the CH1 region lack DNA encoding the hinge region, and expression of the resulting gene may produce a monomeric antibody fragment Fab.

As an example, to prepare a chimeric antibody against the flagellin antigen from P. aeruginosa, SEQ ID No.: 9, encoding the mouse variable heavy chain VH_{M}, may be linked to DNA encoding a human heavy chain constant region CH_{H}. where the DNA encoding the CH_{H} region may encode:
a complete heavy chain constant region, including CH1, CH2, CH3, and a hinge between CH1 and CH2;
a heavy chain constant region fragment, including only CH1 and a hinge linked to CH1; or
a heavy chain constant region fragment, including only CH1.
   SEQ ID No.: 11, encoding the mouse variable light chain VL_{M}, may be linked to DNA encoding a human light chain constant region CL_{H}. The resulting mouse/human chimeric genes are transfected into mammalian cells, and expressed as chimeric anti-FliC antibodies or antibody fragments. Chimeric antibodies against LPS antigens from P. aeruginosa may be prepared in a similar manner.

Fab and F(ab)₂ antibody fragments may also be created by enzymatic digestion of a complete antibody. For example, a chimeric antibody against the flagellin antigen from P. aeruginosa may include:
SEQ ID No.: 9, encoding the mouse variable heavy chain VH_{M}, linked to DNA encoding a human heavy chain constant region CH_{H}. where the DNA encoding the CH_{H} region encodes the CH1, CH2, CH3, and hinge regions; and
SEQ ID No.: 11, encoding the mouse variable light chain VL_{M}, may be linked to DNA encoding a human light chain constant region CL_{H}.

The resulting mouse/human chimeric genes are transfected into mammalian cells, and expressed as chimeric anti-FliC antibodies. The anti-FliC antibodies are recovered, and enzymatically digested to produce antibody fragments. Enzymatic digestion of the chimeric anti-FliC antibodies with pepsin produces F(ab)₂ fragments. Digestion with pepsin cleaves the CH2 and CH3 domains from each heavy chain in the antibody. The heavy chain retains the CH1 and hinge domains. Since the hinge domains on two antibodies are linked by disulfide bonds in a mature antibody, digestion with pepsin produces a dimer with two linked antigen-binding Fab fragments. The F(ab)₂ fragment has a molecular weight of approximately 110 kDa.

Enzymatic digestion of the chimeric anti-FliC antibodies with papain produces two monomeric Fab fragments, where each Fab fragment is a 50 kDa antibody fragment. Digestion with papain cleaves the hinge, CH2 and CH3 domains from each heavy chain in the antibody. The heavy chain retains the CH1 domain. The CH1 domain and the human light chain constant region CL_{H} are connected by disulfide bonds, so each Fab fragment includes a complete antigen-binding domain. Since the Fab fragments lack a hinge region, the heavy chains on the Fab fragments do not form dimers.

Single chain variable fragments (scFV) may also be constructed by connecting a DNA sequence encoding a mouse variable light chain VL_{M} to a DNA sequence encoding a mouse heavy chain variable region VH_{M} through a sequence encoding a peptide linker LINK to produce a fragment lacking a constant region, with the structure VL_{M}-LINK-VH_{M} or VH_{M}-LINK-VL_{M}. The linker is long enough, e.g., 15-20 amino acids long, to allow VL_{M} and VH_{M} to fold together to form a functional antigen-binding domain.

Thus, for example, to prepare an scFV against the flagellin antigen from P. aeruginosa, SEQ ID No.: 9, encoding the mouse variable heavy chain VH_{M} from a mouse anti-FliC antibody, may be linked to SEQ ID No.: 11, encoding the anti-FliC mouse variable light chain VL_{M}, through an intervening DNA sequence encoding a linker peptide sequence LINK containing polyglycine and/or polyserine sequences and intervening hydrophilic amino acids. The resulting scFV might have the structure:

SEQ ID No.: 9-LINK-SEQ ID No.: 11

Bifunctional antibodies may be produced by linking two scFV fragmentss through a second linker (LINK2), where the scFV fragments may be the same or different. Thus, for example, a bifunctional anti-FliC scFV (sc (FV) 2) fragment might have the structure:

SEQ ID No.: 9-LINK-SEQ ID No.: 11-LINK2-SEQ ID No.: 9-LINK-SEQ ID No.: 11

Returning to the concept of bispecific antibodies and bispecific antibody fragments, bispecific antibodies may be constructed by:
connecting a DNA sequence encoding a first mouse variable light chain VL1 to a DNA sequence encoding a first mouse heavy chain variable region VH1 through a sequence encoding a peptide linker LINK to produce a first scFV fragment lacking a constant region, with the structure VL1-LINK-VH1;
connecting a DNA sequence encoding a second mouse variable light chain VL2 to a DNA sequence encoding a second mouse heavy chain variable region VH2 through a sequence encoding a peptide linker LINK to produce a second scFV fragment lacking a constant region, with the structure VL2-LINK-VH2; and
connecting the first and second scFV fragments through a linker LINK2 to produce a bifunctional scFV fragment.

Thus, for example, to prepare a bispecific sc (FV) 2 fragment against both the flagellin antigen and the LPS antigen from P. aeruginosa, an anti-FliC fragment is created by linking SEQ ID No.: 9, encoding the mouse variable heavy chain VH_{M} from a mouse anti-FliC antibody, to SEQ ID No.: 11, encoding the anti-FliC mouse variable light chain VL_{M}, through the intervening DNA linker sequence LINK. An anti-LPS fragment is created by linking SEQ ID No.: 1, encoding the mouse variable heavy chain VH_{M} from a mouse WVDC-0357 (S1F9) antibody, to SEQ ID No.: 3, encoding the WVDC-0357 (S1F9) mouse variable light chain VL_{M}, through the intervening DNA linker sequence LINK. The resulting fragments are linked through a linker LINK2 to produce a bispecific sc (FV) 2 fragment with the structure:

SEQ ID No.: 9-LINK-SEQ ID No.: 11-LINK2-SEQ ID No.: 1-LINK-SEQ ID No.: 3

In various embodiments, a humanized antibody may be made by: replacing the light variable chain CDRs from a human immunoglobulin IgG antibody with CDRs from a variable light chain from a mouse (VL_{M}); and replacing the heavy variable chain CDRs from a human immunoglobulin IgG antibody with CDRs from a variable heavy chain from a mouse (VL_{M}).

For example, to prepare a humanized antibody against the flagellin antigen from P. aeruginosa, CDRs from SEQ ID No.: 10 may be used to replace CDRs in the human heavy chain variable region VH_{H} in a human IgG. CDRs from SEQ ID No.: 12 may be used to replace CDRs in the human light chain variable region VL_{H} in the human IgG.

Table 4 shows human antibody variable chain domains, derived from a partial human immunoglobulin heavy chain variable region having SEQ ID No. 19 and a human immunoglobulin kappa light chain variable region having SEQ ID No. 20 by known methods. In SEQ ID No.: 13, a humanized WVDC-2109 heavy chain is derived by replacing the CDR regions of SEQ ID No.: 19 with CDRs from SEQ ID No. 10. A humanized WVDC-2109 light chain is derived by replacing the CDR regions of SEQ ID No.: 20 with CDRs from SEQ ID No. 12. In Table 4, the human portions of SEQ ID Nos.: 19 and 20 are shown in italic text, while the CDRs are shown in bold underlined text. Humanized light and heavy chains may be derived from WVDC-0357 and WVDC-0496 antibodies in a similar manner; the sequences of such chains are also shown in Table 4.

TABLE 4

| Antibody Chain | Sequence |
|---|---|
| SEQ. ID No. 19: | QVQLVQSGAEVKKPGASVKVSCKASGYTFTSYYMH |
| partial human | WVRQAPGQGLEWMGIINPSGGSTSYAQKFQG |
| immunoglobulin | RVTMTRDTSTSTVYMELSSLRSEDTAVYYCARDP |
| heavy chain | RGGYGSGANYYYYYGMDVWGQGTTVTVSS |
| variable region | |
| SEQ. ID No. 20: | DIQMTQSPSSLSASVGDRVTITCRASQSVRKYLN |
| Partial human | WYQQKPGKAPKLLIYASSLQSGVPSRFSGSGSGT |
| immunoglobulin | DFTLTISSLQPEDFATYYCQQSNSTPWTFGQGTK |
| light chain | VEIKRTVA |
| variable region | |
| SEQ. ID No. 13: | QVQLVQSGAEVKKPGASVKVSCKASGYTFTSYWMH |
| Humanized | WVRQAPGQGLEWMGMIRPNSGSTNYNEKFKSRVT |
| WVDC-2109 | MTRDTSTSTAYMELSSLRSEDTAVYYCARRGYGSSY |
| Heavy chain | GYYAMDYWGQGTTVTVSS |
| SEQ. ID No. 14: | DIQMTQSPSSLSASVGDRVTITCRASQDISNYLN |
| Humanized | WYQQKPGKAPKLLIYYTSRLNSGVPSRFSGSGSGT |
| WVDC-2109 | DFTLTISSLQPEDFATYYCQQGNTLPWTQGTKVEIK |
| Light chain | |
| SEQ. ID No. 15: | QVQLVQSGAEVKKPGASVKLSCKASGYTFTNYWMH |
| Humanized | WVRQAPGQGLEWMGNIDPSDSETHYIQKFKDRVTLT |
| WVDC-0357 | ADTSTSTAYMELSSLTSEDTAVYYCARGGYGSGYAM |
| Heavy chain | DCWGQGTTVTVSS |
| SEQ. ID No. 16: | DIVMTQTPLSLPVTLGQPASISCRSSQSLENSNGNTY |
| Humanized | LNWYLQKPGQSPQLLIYRVSIRFCGVPDRFSGSGSG |
| WVDC-0357 | TDFTLKISRVEAEDVGVYYCLQVTHVPYTFGQGTKLEIK |
| Light chain | |
| SEQ. ID No. 17: | QVQLVQSGAEVKKPGASVKLSCKASGYTFTNYWMHWV |
| Humanized | RQAPGQGLEWMGNIDPSDSETHYIQKFKDRVTLTADTST |
| WVDC-0496 | STAYMELSSLTSEDTAVYYCARGGYGSGYAMDCWGQGTTVTVSS |
| Heavy chain | |
| SEQ. ID No. 18: | DIVMTQTPLSLPVTPGQPASISCRSSQSLENSNGNTYLNW |
| Humanized | YLQKPGQSPQLLIYRVSNRFCGVPDRFSGSGSGTDFTLKIS |
| WVDC-0496 | RVEAEDVGVYYCLQVTHVPYTFGQGTKLEIK |
| Light chain | |

mRNA and DNA Administration Platforms

In various embodiments, preventatives or treatments based on mRNA or DNA may be constructed against P. aeruginosa LPS or flagellin.

mRNA constructs are generated as RNA chains including the following:
A 5′Cap: A 7-methylguanosine nucleotide connected to a triphosphate chain, in turn connected to
A 5′ untranslated region (5′ UTR), which contains an initiation codon, in turn connected to:
An open reading frame (ORF) encoding a gene of interest, in turn connected to:
A 3′ untranslated region (3′ UTR), which follows a termination codon, in turn connected to:
A poly-A (poly-adenosine) tail.

The mRNA construct may be administered in lipid nanoparticles to a patient needing vaccination against, or treatment for, infection by P. aeruginosa LPS or flagellin. Suitable lipids for use in such nanoparticles include ionizable lipids, cholesterol, phospholipids, and PEGylated lipids. Ionizable lipids allow stable complex formation between the lipid and the mRNA.

In various embodiments, a chimeric antibody against the flagellin antigen from P. aeruginosa may be constructed from:
an RNA sequence complementary to SEQ ID No.: 9, encoding the mouse variable heavy chain VH_{M}, linked to RNA encoding a human heavy chain constant region CH_{H}; and
an RNA sequence complementary to SEQ ID No.: 11, encoding the mouse variable light chain VL_{M}, linked to RNA encoding a human light chain constant region CL_{H}.
   The resulting mouse/human chimeric RNA sequences are used as ORF sequences, and are inserted into mRNA sequences between the 5′ and 3′ UTR sequences. The resulting mRNA sequences may be incorporated into lipid nanoparticles, and administered to a patient needing vaccination against, or treatment for, infection by P. aeruginosa flagellin. The mRNA construct stimulates expression of chimeric anti-FliC antibodies.

A DNA construct may be generated from an adenoviral vector, including, for example:
the DNA sequence of SEQ ID No.: 9, encoding the mouse variable heavy chain VH_{M}, linked to DNA encoding a human heavy chain constant region CH_{H}; and
the DNA of SEQ ID No.: 11, encoding the mouse variable light chain VL_{M}, linked to DNA encoding a human light chain constant region CL_{H}.
   Each of these DNA sequences is under the control of a promoter.

A replication-incompetent adenovirus containing the resulting vector may be administered to a patient needing vaccination against, or treatment for, infection by P. aeruginosa flagellin. The DNA construct containing the vector stimulates expression of chimeric anti-FliC antibodies.

mRNA and DNA construct containing nucleic acid sequences encoding humanized anti-P. aeruginosa FliC antibodies may be constructed in a similar fashion. mRNA and DNA constructs containing nucleic acid sequences encoding humanized or chimeric anti-P. aeruginosa FliC antibodies may be constructed in a similar fashion.

### Drug-Antibody Conjugates

In various embodiments disclosed herein, drugs may be conjugated to the monoclonal antibodies WVDC-2109, WVDC-0496 (S3D4), WVDC-0357 (S1F9), as well as to chimeric, humanized, or bispecific antibodies derived from WVDC-2109, WVDC-0496 (S3D4), or WVDC-0357 (S1F9) variable regions. The resulting antibody-drug conjugates may be for administration to a patient. The drug may be a small molecule antimicrobial or antibiotic drug, The drug may be:
a penicillin type antibiotic, e.g., penicillin, amoxicillin, co-amoxiclav, flucloxacillin, or phenoxymethylpenicillin;
a macrolide type antibiotic, e.g., azithromycin, clarithromycin, fidaxomicin, or erythromycin;
a cephalosporin type antibiotic, e.g., cefepime, cefpirome, cefotetan, cefoxitin, cefprozil, cefuroxime, loracarbef, ceftazidime, cefoperazone, or ceftaroline; or
a fluoroquinolone type antibiotic, e.g., ciprofloxacin, gemifloxacin, levofloxacin, moxifloxacin, or ofloxacin.

In various embodiments, a cleavable linker is used to connect the drug to the antibody. The linker may have a hydroxy, carboxy, or amino group which may be condensed with an appropriate functional group on the drug. The linker may also have a maleimide group, where the maleimide group reacts with a thiol moiety on the antibody. The linker may have a chain with a cleavable bond, such as:
a disulfide bond, which is cleaved by intracellular reducing agents, e.g., glutathione;
a hydrozone bond, which is cleaved by acidic intracellular pH values; or
a peptide bond, e.g., Val-Cit or Val-Ala, which is cleaved by intracellular enzymes.

The resulting drug-antibody conjugate may be administered to a patient infected by a bacteria. The antibody binds the bacteria, and the linker is cleaved, allowing the antibiotic drug to enter the cell.

### EXAMPLES

In the following examples, all bacterial strains used are listed in Table 2. All Pseudomonas aeruginosa strains were grown in Luria-Bertani broth (LB) or on Pseudomonas Isolation Agar (PIA) at 37° C. Other bacterial species were cultured in Tryptic Soy Broth (TSB) or on TS agar (TSA) plates at 37° C.

In the following examples, all statistical analyses were performed using GraphPad Prism version 9. Survival data is presented as a Kaplan-Meier curve and analyzed by the log-rank (Mantel-Cox) test. All other data is presented as the mean+standard deviation (SD). For comparison between two groups, a two-tailed student's t-test was performed. For comparison between three or more groups, a one-way ANOVA with Dunnett's multiple comparison or Kruskal-Wallis with Dunn's multiple comparison was performed depending on the normality of the data set. P values less than 0.05 were considered statistically significant.

### P. Aeruginosa Lipopolysaccharide (Lps) Antibodies

### Example 1. Generation of Hybridomas

Six-week-old CD-1 mice (Charles River, strain 022) were immunized twice with an intranasal dose of 1×10⁷ CFU of live P. aeruginosa strain PAO1 Vasil grown on Pseudomonas isolation agar (PIA) supplemented with 0.27 mM ammonium metavanadate (AMV). Hybridomas were generated by fusing 1×10⁷ splenocytes from an immunized mouse with 1×10⁷ SP2/O-Ag14 myeloma cells (ATCC CRL-1581™) using the ECM2001+electrofusion apparatus (BTX). Fused cells were cultured in ClonaCell-HY medium C (STEMCELL Technologies) and incubated overnight at 37° C. and 5% CO₂. The next day, cells were centrifuged and cultured in ClonaCell-HY medium D (STEMCELL Technologies), which is a semi-solid medium containing hypoxanthine-aminopterin-thymidine (HAT). After ten days of incubation, single hybridoma colonies were selected and plated in 96 well plates containing ClonaCell-HY medium E (STEMCELL Technologies). Supernatant from the well plates were screened with ELISA to identify IgG antibodies against P. aeruginosa. Select hybridoma colonies were produced at high densities using a hollow fiber bioreactor (FiberCell Systems) containing Dulbecco's Modified Eagle's Medium (DMEM) supplemented with serum-free chemically defined medium for high density cell culture (CDM-HD). Monoclonal antibodies from the bioreactor harvests were purified with the ÄKTA pure chromatography system (Cytiva) using a HiScreen Fibro™ Prism A column (Cytiva) and HiScale™ 16/20 column (Cytiva). Purified monoclonal antibodies were eluted in 0.2M sodium phosphate buffer (pH 7.4) and antibody concentrations were determined by measuring the Protein A280 with an E1% value of 14.00. The isotype of each antibody was determined using the Pierce Rapid Antibody Isotyping Kit (ThermoFisher Scientific, #26179).

### Example 2. Enzyme-Linked Immunosorbent Assay for Binding to P. aeruginosa

P. aeruginosa strain PAO1 Vasil and cystic fibrosis P. aeruginosa isolates, shown in Table 2, were grown on PIA overnight at 37° C. Bacterial plates were swabbed into sterile PBS and adjusted to a bacterial density that corresponded to 1×109 CFU/ml. To prepare ELISA plates, 96-well microtiter plates (ThermoFisher Scientific, #15041) were coated with 50 μL of bacteria containing 5×10⁷ CFU in PBS and incubated overnight at 4° C. Wells were washed three times with PBS with 0.05% Tween 20 (PBS-T) and blocked with 2% bovine serum albumin (BSA) (Research Products International, #A30075) overnight at 4° C. Primary antibodies (serum, hybridoma supernatant, mAb WVDC-0357 (S1F9), or mAb WVDC-0496 (S3D4)) were diluted in 2% BSA were applied to the wells and incubated for 1h at 37° C. Wells were washed four times with PBS-T. For plates developed with tetramethylbenzidine (TMB), 100 μl of anti-IgG secondary antibody conjugated to horseradish peroxidase (HRP) (Novus biological, #NBP1-75130) at a dilution of 1:2000 in 2% BSA was applied to each well for 1h at 37° C. After incubation, wells were washed five times with PBS-T and 100 μL of a TMB solution (BioLegend, #421101) was added to each well. After 10-15 min, the reaction was stopped by adding 50 ul of 2N sulfuric acid and absorbance was read at 450 nm using a SpectraMax i3 plate reader (Molecular Devices LLC). For plates developed with Pierce p-Nitrophenyl Phosphate (PNPP), 100 μl of anti-IgG secondary antibody conjugated to alkaline phosphatase (AP) (Southern Biotech, #1030-04) at a dilution of 1:2000 in 2% BSA was applied to each well for 1h at 37° C. After incubation, wells were washed five times with PBS-T and 100 μL of a PNPP solution (ThermoFisher Scientific, #34045) was added to each well. After 30 min, the absorbance was read at 405 nm using a SpectraMax i3 plate reader.

### Example 3. Western Blot

To identify the target of antibody binding, western blot analysis was performed with PAO1 bacterial lysates treated with proteinase K and sodium periodate. To prepare bacterial cell lysates, PAO1 Vasil was grown on PIA overnight at 37° C., swabbed and resuspended into PBS, and lysed with sonication. Bacterial lysates were treated with 0.2 mg/ml proteinase K (Invitrogen, #25530049) or with 20 mM sodium periodate (ThermoFisher Scientific, #20504). Bacterial cell lysate was also prepared with strains from the University of Washington PAO1 transposon mutant library. Strains from the transposon mutant library were grown on lysogeny agar (LA-Miller) overnight at 37° C., swabbed and resuspended into PBS, and lysed with sonication. After lysate preparation, 2 μg of lysate was added to Laemmli buffer (Sigma-Aldrich, #S3041) and boiled at 95° C. for 5 min. Samples were then loaded into 12% tris-glycine gels (ThermoFisher, #XP00122BOX) and resolved by gel electrophoresis. Samples were transferred onto nitrocellulose membranes (ThermoFisher, #IB23001) using the iBlot™ 2 Gel Transfer Device (ThermoFisher, #IB21001) and blocked overnight in 5% skim milk. Membranes were treated with 2 μg/ml of mAb WVDC-0357 (S1F9) or mAb WVDC-0496 (S3D4) in 1% skim milk for one hour at room temperature. Membranes were then washed three times with PBS-T and treated with anti-IgG secondary antibody conjugated to HRP (Novus biological, #NBP1-75130) at a 1:5000 dilution in 1% skim milk for one hour at room temperature. Membranes were washed three times with PBS-T and developed using Pierce™ ECL Western Blotting Substrate (ThermoFisher, #32106). Chemiluminescent images of the blots were taken using a Chemidoc Touch Imaging System (Bio-Rad, #1708370).

### Example 4. Flow Cytometry Binding Assay

P. aeruginosa strain PAO1 was cultured to mid-log phase growth, washed, and concentrated in PBS. Approximately 1×10⁷ CFUs of bacteria were incubated with 10 μg/ml of antibody for 30 min at 37° C. Samples were washed once with PBS and incubated with 1.25 μg/ml Fc block (BD Biosciences, #553142) for 15 min at 4° C. Afterwards, 2.5 μg/ml of anti-IgG secondary antibody conjugated to APC-Cy7 (BioLegend, #405316) was added to samples and incubated for 30 min at 4° C. Samples were washed twice with PBS and P. aeruginosa was stained with BacLight Green bacteria stain (Invitrogen, #B35000). Samples were analyzed on a Guava® easyCyte 12HT flow cytometer (Luminex). Flow cytometry results were analyzed using FlowJo™ v10.8 Software (BD Life Sciences).

### Example 5. Bacterial Killing Assay

To assess the capability of mAbs to facilitate clearance of bacteria, a modified version of an opsonophagocytosis killing assay was performed as described previously. P. aeruginosa strain PAO1 was cultured to mid-log phase growth, washed, and resuspended to 1×104 CFU/ml in opsonization buffer B (OBB; phosphate buffered saline supplemented with 5% heat-inactivated FBS and 0.1% gelatin). In round bottom 96-well plates (Fisherbrand, #FB012932), 50 μl of bacteria and 50 μl of treatment (PBS, isotype control mAb, mAb WVDC-0357 (S1F9), or mAb WVDC-0496 (S3D4)) were added to each well and incubated for one hour at room temperature with shaking. Afterwards, J774A.1 macrophages (ATCC, #TIB-67™), baby rabbit complement (Pel-Freez Biologicals, #31061), J774A.1 macrophages with baby rabbit complement, or OBB was added to each well and incubated for one hour at 37° C. with shaking. A 1000:1 effector-to-target cell ratio of macrophages to bacterium was used. After incubation, plates were put on ice for 15 minutes to stop the reaction. Samples were serially diluted and plated on PIA. Plates were incubated overnight at 37° C. Remaining bacterial colonies were enumerated and expressed as percent killings relative to assays run without antibody.

### Example 6. Agglutination Assay

In UV-transparent cuvettes (BrandTech, #759015) and culture tubes, an overnight culture of PAO1 was diluted 1:50 in Lysogeny broth and treated 100 μg/ml of isotype control, WVDC-0357 (S1F9), or WVDC-0496 (S3D4) mAb. Cuvettes were incubated statically at 37° C. for 24 h and the absorbance at 600 nm was read at the time points up to 24 h. To visualize bacterial agglutination, PAO1 was cultured to mid-log phase growth, washed, and concentrated in PBS. Approximately 5×10⁷ CFUs of bacteria was stained with BacLight Red bacterial stain (Invitrogen, #B35001) for 15 minutes at room temperature, pelleted, and resuspended in PBS. Antibody was added at a final concentration of 100 μg/ml and incubated at 37° C. for 2 h. Samples were placed on glass slides, mounted with cover slips, and sealed with nail polish. Fluorescent images were taken using an EVOS FL imaging system (ThermoFisher, #AMEFC4300). For aggregation analysis, 20 randomly imaged areas were analyzed for number and size of aggregates using a custom pipeline in CellProfiler.

### Example 7. Murine Challenge Models

To examine protection in PAO1-PIAAMV convalescent mice, a murine acute pneumonia model was used. P. aeruginosa strain PAO1 was cultured on PIAAMV overnight at 37° C. Bacterial plates were swabbed and resuspended in PBS. Based on the optical density at 600 nm, a dose was prepared at 1.35×10⁷ CFU per 20 μL. Six-week old, female CD-1 mice were anesthetized with ketamine (77 mg/kg) (Patterson Veterinary, #07-803-6637) and xylazine (7.7 mg/kg) (Patterson Veterinary, #07-909-1939) in 0.9% saline. Anesthetized mice were intranasally infected by pipetting 10 μL of bacteria in each nostril. Mice were allowed to convalesce for 34 days. A dose of PAO1 cultured on PIA was prepared at 1.4×10⁸ CFU per 20 μL. Convalescent mice and age-matched naïve CD-1 mice were anesthetized and intranasally infected with the prepared dose. Mice were euthanized at 16 h post-infection with an IP injection of Euthasol (390 mg/kg) (Patterson Veterinary, #07-805-9296). Blood was collected via cardiac puncture and placed in serum separator tubes (BD, #365967). The lung was aseptically removed, homogenized, and plated to determine CFU loads. The nasal cavity was flushed with PBS and plated to determine CFU loads in the nares.

To examine the efficacy of mAbs WVDC-0357 (S1F9) and WVDC-0496 (S3D4) in a sepsis model of infection, nine-week old female CD-1 mice were IP administered an IgG2b isotype control antibody (BioXcell, #BE0086), P. aeruginosa whole-cell vaccinated serum (Pa WCV), mAb WVDC-0357 (SIF9), or mAb WVDC-0496 (S3D4). Twelve hours after administration, mice were infected with an IP 5×10⁵ CFU dose of PAO1. To determine survival, mice were monitored for up to 5 days after infection. The health of the mice were scored based on six criteria: appearance, activity, eye closure, respiration quality, body temperature, and body weight loss. For each category, mice were scored from 0-4, where 0 represented no symptoms, and 4 represented the most severe phenotype. Mice were humanely euthanized if mice reached a score of 4 in any category or total score of 12 and above.

To look at correlates of protection in the sepsis model, mice were passively immunized as before, and euthanized at 6h post-infection for tissue collection. Mice were euthanized with an IP injection of Euthasol (390 mg/kg). Blood was collected via cardiac puncture and placed in serum separator tubes. The kidney and spleen were aseptically removed. The left kidney and half the spleen were placed in 10% neutral buffered formalin and sent for histopathological analysis. The right kidney was Dounce homogenized and remaining spleen was homogenized in a gentleMACS C tube (Miltenyi Biotec, #130-093-237). To quantify bacterial burden, blood, kidney and spleen samples were serially diluted in PBS, plated on PIA, and incubated overnight at 37° C. for CFU enumeration.

To examine the efficacy of mAbs WVDC-0357 (SIF9) and WVDC-0496 (S3D4) in an acute pneumonia murine model of infection, six-week old female CD-1 mice were IP administered either an IgG2b isotype control mAb, Pa WCV serum, mAb WVDC-0357 (S1F9), or mAb WVDC-0496 (S3D4). Twelve hours after administration, mice were intranasally infected with a 2×10⁷ CFU dose of PAO1. Mice were euthanized at 16h post-infection with an IP injection of Euthasol (390 mg/kg). Blood was collected via cardiac puncture and placed in serum separator tubes. The lung was aseptically removed and weighed. The left lobe of the lung was placed in 10% neutral buffered formalin and sent for histopathological analysis. The right lobe of the lung was placed in a gentleMACS C tube and homogenized. The nasal wash was collected by injecting 1 mL of PBS through the nasal cavity. The homogenized lung and nasal wash were serially diluted, plated on PIA, and incubated overnight at 37° C. for CFU enumeration.

The concentration of CXCL-1, TNF-α, IL-βB, IL-6, IL-10, and IFN-γ in the serum and lung supernatant was quantified using a Luminex multiplex assay (R&D). Procedures were performed according to the manufacturer's instructions.

All animal care and use was in compliance with the National Institutes of Health Guide for the Care and Use of Laboratory animals. The animal protocols used in this study were approved by the West Virginia University Institutional Animal Care and Use Committee (WVU-ACUC protocol 1606003173).

### Example 8. Antibody Sequencing

The variable regions of WVDC-0357 (SIF9) and WVDC-0496 (S3D4) were sequenced using methods known in the art. Total RNA was extracted using Quick-RNA Microprep Kit (Zymo, #R1051). The cDNA was synthesized using the SMARTScribe Reverse Transcriptase kit (Clontech, #639537) with a custom template switch oligonucleotide and chain-specific reverse primers. Touchdown PCR was used to amplify the synthesized cDNA with a universal forward primer and nested chain-specific reverse primers. The amplified PCR reaction was run on a 1% agarose gel and DNA was extracted using the QIAquick Gel Extraction Kit (Qiagen, #28704). The extracted DNA was Sanger sequenced using the IgG reverse primers.

### P. Aeruginosa and B. Pseudomallei Flagellin (Flic) Antibodies

### Example 9. Generation of Hybridomas

In order to generate hybridomas, six week old CD-1 female mice were immunized intraperitoneally with 50 μg of CRM 197 conjugated FliC peptide, consisting of amino acids 51-90 from B. pseudomallei FliC sequence, formulated 1:1 with Complete Freund's adjuvant. At day 21, mice were boosted with 50 μg of conjugated FliC peptide adjuvanted with Incomplete Freund's adjuvant. Mice were bled 1 week after each injection. The polyclonal antibody response was evaluated by enzyme-linked immunosorbent assay (ELISA) using conjugated and non-conjugated peptides, P. aeruginosa PAO1 and B. pseudomallei Bp82 coated plates.

Thirty-four days post initial infection, the mouse with the highest immune response was euthanized with CO₂. Following euthanasia, blood was collected via cardiac puncture and splenocytes and inguinal lymph nodes were taken and fused with P3X63Ag8.653 myeloma cells (ATCC®: CRL-1580™); 10⁷ myeloma cells were mixed with 10⁷ splenocytes and inguinal lymph nodes cells in serum free Medium B (ClonaCell™-HY Medium B, #03802, StemCell Technologies). The mixture was centrifuged and washed 3 times with electrofusion medium (Btxpress Cytofusion Medium C, #47001, BTX®). After the last wash, cells were placed into a BTX® coaxial chamber and subjected to electrofusion using an ECM 2001 Electrofusion and Electroporation System (BTX®). After fusion, cells were kept undisturbed into the fusion chamber for 5 min and transferred to a 75 cm2 cell culture treated flask containing Medium C (ClonaCell™-HY Medium C, #03803, StemCell Technologies). Flasks were incubated at 37° C., 5% CO₂, 16-24 h after what cells were centrifuged, resuspended with 10 mL of Medium C and transferred to 90 mL of semi solid Medium D (ClonaCell™—HY Medium D, #03804, StemCell Technologies). After 15 minutes of incubation at room temperature, 9.5 mL of the suspension were plated into 100 mm×15 mm Petri dishes using a 10 ml syringe with a 16-gauge blunt-end needle. Plates were incubated at 37° C., 5% CO₂. Clones representing hybridomas producing monoclonal antibodies were obtained after approximately 10 days. Clones were then individually transferred into 96 well tissue cultured treated plates containing Medium E (ClonaCell™-HY Medium E, #03805, StemCell Technologies). After 3-4 days, supernatants of the hybridoma cells were removed and tested by ELISA for antigen specificity against conjugated FliC peptide. Positive hybridomas were selected and transferred to 24-well plates containing Medium E. Once confluent, hybridoma supernatants were tested again by ELISA for their reactivity against conjugated and non-conjugated FliC peptide, P. aeruginosa PAO1 and B. pseudomallei Bp82. Positive hybridomas were selected and expanded into 50% Medium A (ClonaCell™-HY Medium A, #03801, StemCell Technologies) and 50% of Dulbecco's Modified Eagle Medium (DMEM) (Thermo Fisher Scientific, #MT10013CM) supplemented with 10% v/v Fetal Bovine Serum (FBS) (Gibco, #10437028) and 1% v/v Penicillin-Streptomycin Solution (P/S) (Corning cellgro, #30-001-Cl).

The antibody class and subclass and the light chain type were determined by using Pierce™ Mouse Antibody Isotyping Kit (#26178, Thermo Scientific).

### Example 10. ELISA

ELISA studies were performed by known methods. 96-well microtiter plates were coated overnight at 4° C. with different antigens:
1 μg/ml of CRM-conjugated FliC peptide;
1 μg/ml non-conjugated FliC peptide;
1 μg/ml of recombinant FliC from P. aeruginosa or B. pseudomallei; or
2-5×10⁷ CFU of the pathogens of interest grown overnight on LB or TSB.

Plates were blocked with 2% w/v bovine serum albumin (BSA) in PBS overnight at 4° C. Hybridoma supernatant or purified WVDC-2109 mAb were serially diluted in 2% BSA and incubated 2 h at 37° C. After four washes with 0.05% Tween in PBS (PBS-T), horseradish peroxidase conjugated mouse anti-IgG (Novus Biologicals, #NBP1-75130) was added to the plate at a dilution of 1:2000 for 1 h at 37° C. Finally, a 1:1 mixture of TMB substrates A (Biolegend, #77247) and B (Biolegend, #77248) was used as a substrate. After 15 min of incubation, the absorbance was measured at 450 nm using SpectraMax i3 spectrophotometer (Molecular Devices LLC). Antibody titers were calculated as the highest dilution at which the absorbance of the sample exceeded two times the absorbance of the negative control.

### Example 11. Recombinant Flagellin Purification

The fli (′ genes from B. pseudomallei strain Bp82 and P. aeruginosa PAO1 were amplified and cloned into pHERD plasmid with a polyhistidine-tag sequence added. Fli (′ genes were expressed in E. coli E. cloni® Electrocompetent cells (Lucigen), as polyHis-tag FliC protein. Bacterial cells were grown in LB supplemented with Carbenicillin 100 μg/ml. Proteins were purified from the cell culture supernatant using a histidine column (HisPur™ Cobalt Purification Kit, Fisher Scientific, #90092).

### Example 12. Monoclonal Antibody Sequencing

The monoclonal antibody sequence was determined using the protocol described by von Boehmer L et al, with minor modifications. Total RNA was extracted from hybridoma cells using Qiagen RNeasy Mini Kit (Qiagen, #74106). Isolated RNA was treated with DNase using Turbo DNase (Thermo Scientific, AM2238). After DNAse treatment, 100 μg of RNA, 300 ng/μl of random primers (Fisher Scientific, #48-190-011) and 200 U/μl of SuperScript III Reverse Transcriptase (Thermo Fisher Scientific, #18-080-044) were used to synthesize cDNA by reverse transcription (RT). The resulting cDNA was then used to amplify the heavy- and light-domains in two consecutive PCR reactions. For each cDNA sample, three PCR reactions were set up: one for the heavy chain, one for the light kappa chain and one for the light lambda chain. For the first PCR, a forward primer mix, annealing to the V (D) J leader sequences, and a reverse primer, annealing a constant region, were used. The second PCR was performed with a forward primer mix annealing the 5′region of the variable (V) genes and a reverse primer annealing a nested constant region. For both PCRs, HotStar Taq DNA polymerase (Qiagen, #203203) was used.

### Example 13. Monoclonal Antibody Purification

The hybridoma cell line producing WVDC-2109 was cultured in 50% Medium A and 50% DMEM as indicated above. When confluent, cells were transferred into fresh medium at a proper concentration. Supernatants were collected, centrifuged 5 min at 300×g and filtered with 0.2 μm pore-size filter to remove cellular debris. WVDC-2109 was purified by affinity chromatography with a GE AKTA Pure Chromatography System by using Protein A-followed by size-exclusion chromatography. The eluted antibody was dialyzed in PBS for 24 h followed by a 5 h dialysis with 50% Glycerol in PBS and preserved at −20° C. until needed. Prior use, to avoid toxicity effects the excess of glycerol was removed using 50 kDal Amicon Ultra-2 Centrifugal Filter Unit (EMD Millipore, #UFC205024).

### Example 14. Western Blot Analysis

Bacteria of interest were grown in TSA plates overnight at 37° C., swabbed into sterile PBS, incubated on ice for 30 min and lysed using sonication. When necessary, cell lysates were treated with proteinase K (PK) (1.3 mg/ml final concentration) or with sodium periodate (NaIO₄) (13.3 nM final concentration). Protein concentration was determined using Pierce™ BCA Protein Assay Kit (Thermo Fisher, #23225). Recombinant FliC protein from P. aeruginosa and B. pseudomallei were purified as described above. Protein samples were mixed with 4× Laemmli Sample Buffer (Bio-Rad, #1610747) and Thermo Scientific™ Bond-Breaker™ TCEP Solution (Thermo Scientific, #77720) and boiled at 95° C. for 10 minutes. Depending on the analysis, 3 to 10 μg of protein were loaded into protein gels (Bio-Rad, #4569033). Gels were transferred onto rehydrated 0.2 μm PVDF membranes (Bio-Rad, #1704272) using a Trans-Blot Turbo™ Transfer System (Bio-Rad). Membranes were blocked for 1 h in 5% w/v skim milk in PBS-T and incubated overnight at 4° C. with WVDC-2109 mAb at a concentration of 5 μg/ml in blocking solution. Next, blots were treated for 1 h at room temperature with anti-IgG secondary antibody conjugated to HRP (EMD Millipore, #AP127P) at a concentration of 1:5000 in blocking solution. Finally, membranes were developed using ECL Western Blotting Substrate (Thermo Scientific, #32209). Chemiluminescence signal was detected using Chemidoc Touch Imaging System (Bio-Rad, #1708370). Images were visualized and analyzed with Image Lab software version 6.1.0 (Bio-Rad laboratories).

### Example 15. Complement Bactericidal Assay

Complement-mediated bactericidal activity of WVDC-2109 mAb was determined with 30% (v/v) Guinea Pig Complement (MP Biomedicals™, Fisher Scientific, #ICN642831) as a source of complement, 30 μg/ml of WVDC-2109 and 10⁶ CFU/ml P. aeruginosa PAO1 grown in LB, in a final volume of 100 μl. Samples were taken at time 0 and 90 min after incubation at 37° C. In order to determine the viable bacteria at each time point, samples were diluted in PBS and plated on PIA. The percentage of bacterial survival was calculated relative to the number of bacteria at time 0 min.

### Example 16. Macrophage Opsonophagocytosis Assays

Macrophage phagocytosis was determined by using J774A.1 (ATCC®: TIB-67) macrophages. One day before the experiment, 2.5×10⁵ macrophages were incubated in wells of a cell culture-treated 24-well plates while in DMEM supplemented with 10% FBS and 1% of penicillin and streptomycin. The following day, 1-2×10⁶/ml of P. aeruginosa PAO1 from an exponential culture were opsonized with buffer alone or 100 μg/ml of WVDC-2109 for 1 h at 37° C. with slightly agitation. Opsonized bacteria were then centrifuged to remove the unbound antibody, resuspended in DMEM supplemented with 10% FBS and added to each well of the previously washed macrophage plates at multiplicity of infection (MOI) of 10. Plates were centrifuged for 5 min at 250×g and incubated at 33° C. for 15 min and 5% CO₂.

To measure attachment and uptake into macrophages, plates were washed three times with PBS and 1 ml of 0.5% Triton X-100 (Sigma-Aldrich, T8787) was added. Samples were immediately transferred into 1.5 mL tubes, serially diluted and plated on PIA.

To measure the killing mediated by macrophages, after 15 minutes incubation, plates were washed three times and 10 μg/ml of polymixin B was added. Plates were incubated 90 min at 33° C., 5% CO₂, after what were washed thrice and cells were lysed by adding 0.5% Triton X-100. Like for the attachment measurements, samples were immediately transferred into 1.5 mL tubes, serially diluted and plated on PIA. A control without macrophages was added.

### Example 17. TLR5 Assays

TLR5 activation was analyzed using HEK-Blue™-hTLR5 cells (InvivoGen, #hkb-htlr5). Cells were cultivated in DMEM with 4.5 g/l glucose supplemented with 10% FBS (v/v), 1% P/S (v), 100 μg/ml Normocin (InvivoGen, #ant-nr-1), 30 μg/ml Blasticidin (InvivoGen, #ant-b1-1) and 100 μg/ml Zeocin (InvivoGen, #ant-zn-1) until reaching 70-80% of confluence. The day of the experiment, cells were washed with PBS and detached from the flask. A cell suspension of 1.4×10⁵ cells/ml was prepared in HEK-Blue™-Detection medium (InvivoGen, #hb-det).

To test the ability of WVDC-2109 to block flagellin mediated TLR-5 activation, samples were prepared by incubating different concentrations of recombinant purified FliC from P. aeruginosa (0-40 ng/ml) or B. pseudomallei (0-20 ng/ml) with different concentrations of WVDC-2109 (0-100 μg/ml). After 90 min of incubation at 37° C., with slightly agitation, 20 μl were added by duplicate to flat-bottom 96-well plates. 180 μl of the HEK-Blue™-hTLR5 cell suspension was added to each sample and mixed. SEAP activity was measured at 650 nm using SpectraMax i3 spectrophotometer (Molecular Devices LLC) after 16 h of incubation at 37° C. and 5% CO₂.

### Example 18. Monoclonal Antibody Docking

The monoclonal antibody was predicted using the DeepAb antibody structure prediction tool1. The fliC structures for both P. aeruginosa and B. pseudomallei were predicted by AlphaFold2,3. The monoclonal antibodies and fliC protein structures were docked using ClusPro protein-protein docking tool4,5. Antibody mode was used for docking prediction6. The structures were visualized using UCSF ChimeraX7,8.

### Example 19. Mouse Models of Infection

In vivo efficacy of WVDC-2109 mAb was determined in prophylaxis model of sepsis and murine pneumonia. For lethal sepsis model, nine-week-old female outbred CD-1 mice received intraperitoneally 45 mg/kg of WVDC-2109 mAb diluted in PBS or an isotype IgG murine antibody as a negative control. After 12 h, mice were challenged intraperitoneally with 5×10⁶ CFU of P. aeruginosa PAO1 from an exponential culture. Mice were monitored for 7 days (every 2 h for the first 12 h after infection, and every 12 h for the rest of the experiment) to determine the survival rate. Mice were also scored for morbidity attending to six different criteria: 1) appearance, 2) activity, 3) eyes closure, 4) respiration quality, 5) temperature loss and 6) body weight loss. Each variable was given a score from 0 (no symptoms) to 4 (worst symptoms). Mice were euthanized when they were moribund, understanding as reaching a score of 4 in any of the variables or an accumulative score of 12 or above, following the protocols approved by West Virginia University institutional animal use and care committee. Euthanized animals were scored as succumbing to infection at the next health check.

To measure bacterial burden in blood, spleen, and kidneys, mice were immunized and infected as described above. After 6 or 12 h after challenge, mice were euthanized using 390 mg/Kg of pentobarbital (Patterson Veterinary, #07-805-9296). Blood was collected via cardiac puncture. Part of the blood was used to determine CFUs by plating the appropriate dilutions while the rest was centrifuged to obtain the serum. The spleen and kidneys were removed under sterile conditions and homogenized. Bacteria present on those organs were quantified by diluting and plating the samples on PIA. Cytokines present in serum were determined using a Mouse Magnetic Luminex Assay (R&D Systems, #LXSAMSM) following manufacturer's instructions.

For the sublethal pneumonia model, six-week-old female CD-1 mice were treated with 45 mg/kg of WVDC-2109 mAb, an isotype IgG antibody as a negative control or two times the equivalent titers of antibody present in serum from mice previously vaccinated with heat-inactivated P. aeruginosa PAO1 (Pa-WCV) as a positive control. After 12 h, mice were anesthetized with ketamine (77 mg/Kg) (Patterson Veterinary, #07-803-6637) and xylazine (7.7 mg/Kg) (Patterson Veterinary, #07-808-1939) in 0.9% saline and infected through intranasal route with 20 μl (2×10⁷ CFU) of P. aeruginosa PAO1. Approximately 14-16 h after infection, mice were euthanized using 390 mg/Kg of pentobarbital. Blood was collected via cardiac puncture and the serum was obtained by centrifugation and stored until needed. The lungs were removed under sterile conditions and weighted. The right lobes were fixed with formalin 10% (VWR®, #16004-128) for pathological examination. The left lobes were homogenized using GentleMACS™ dissociator (Miltenyi Biotec). Nasal washes were obtained by inserting 1 mL of PBS through the nasal cavity. Bacteria present in the lungs and nasal cavity was quantified by plating serially dilutions on PIA. All plates were incubated overnight at 37° C. before counting CFUs.

Cytokines present in serum (sepsis model) and lung homogenate supernatant (pneumonia model) were determined using a Mouse Magnetic Luminex Assay (R&D Systems, #LXSAMSM).

Fixed samples from the lung were paraffin embedded and cut into 5 μm sections. Histology micrographs were obtained by light microscopy after stained with hematoxylin and eosin.

### CONCLUSION

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A monoclonal antibody against a Gram-negative bacterial pathogen, comprising:
a first variable region with:
a heavy chain having a protein sequence of SEQ. ID No. 2 or SEQ. ID No. 6; and
a light chain having a protein sequence of SEQ. ID No. 4 or SEQ. ID No. 8; or
a second variable region with:
a heavy chain having a protein sequence of SEQ ID. No. 10; and
a light chain having a protein sequence of SEQ ID. No. 12.

2. A monoclonal antibody against a Gram-negative bacterial pathogen, comprising:
a first variable region with:
a heavy chain having a first CDR comprising residues 79-83 of SEQ. ID No. 2 or residues 78-82 of SEQ. ID No. 6; a second CDR comprising residues 98-114 of SEQ. ID No. 2 or residues 97-113 of SEQ. ID No. 6; and a third CDR comprising residues 147-157 of SEQ. ID No. 2 or residues 146-156 of SEQ. ID No. 6; and
a light chain having a first CDR comprising residues 69-84 of SEQ. ID No. 4 or SEQ. ID No. 8; a second CDR comprising residues 100-10⁶ of SEQ. ID No. 4 or SEQ. ID No. 8; and a third CDR comprising residues 139-147 of SEQ. ID No. 4 or SEQ. ID No. 8; or
a second variable region with:
a heavy chain having a first CDR comprising residues 31-35 of SEQ. ID No. 10; a second CDR comprising residues 50-66 of SEQ. ID No. 10; and a third CDR comprising residues 99-112 of SEQ. ID No. 10; and
a light chain having a first CDR comprising residues 24-34 of SEQ. ID No. 12; a second CDR comprising residues 50-56 of SEQ. ID No. 12; and a third CDR comprising residues 89-97 of SEQ. ID No. 12.

3. The monoclonal antibody of claim 1, wherein the monoclonal antibody has the first variable region, and is configured to bind to lipopolysaccharides of serogroup O5 strains of P. aeruginosa.

4. The monoclonal antibody of claim 1, wherein the monoclonal antibody has the second variable region, and is configured to bind to a flagellin protein of the Gram-negative bacterial pathogen.

5. The monoclonal antibody of claim 4, wherein the Gram-negative bacterial pathogen is selected from the group consisting of P. aeruginosa, Burkholderia pseudomallei, Burkholderia thailandensis, Burkholderia cepacian, Burkholderia cenocepacia, Klebsiella pneumoniae, Serratia marcescens, Enterobacter cloacae, Salmonella enterica, Acinetobacter baumannii, Escherichia coli, and Bordetella pertussis.

6. The monoclonal antibody of claim 2, wherein the monoclonal antibody has the first variable region, and is configured to bind to lipopolysaccharides of serogroup O5 strains of P. aeruginosa.

7. The monoclonal antibody of claim 2, wherein the monoclonal antibody has the second variable region, and is configured to bind to a flagellin protein of the Gram-negative bacterial pathogen.

8. The monoclonal antibody of claim 7, wherein the Gram-negative bacterial pathogen is selected from the group consisting of P. aeruginosa, Burkholderia pseudomallei, Burkholderia thailandensis, Burkholderia cepacian, Burkholderia cenocepacia, Klebsiella pneumoniae, Serratia marcescens, Enterobacter cloacae, Salmonella enterica, Acinetobacter baumannii, Escherichia coli, and Bordetella pertussis.

9. The monoclonal antibody of claim 2, wherein the monoclonal antibody is humanized.

10. A bispecific antibody or antibody fragment against a Gram-negative bacterial pathogen, comprising:
a first variable region with:
a heavy chain from a first antibody against a lipopolysaccharide from the Gram-negative bacterial pathogen linked to a light chain from a second antibody; and
a heavy chain from the second antibody linked to a light chain from the first antibody against the lipopolysaccharide from the Gram-negative bacterial pathogen.

11. The bispecific antibody or antibody fragment of claim 10, wherein the second antibody binds a flagellin protein.

12. A method of treating a patient,
wherein the patient is infected with a Gram-negative bacterial pathogen, or at risk of being infected with the Gram-negative bacterial pathogen;
wherein the Gram-negative bacterial pathogen is a serogroup O5 strain of P. aeruginosa,
the method comprising administering an effective amount of the monoclonal antibody of claim 3 to the patient.

13. A method of treating a patient,
wherein the patient is infected with a Gram-negative bacterial pathogen, or at risk of being infected with the Gram-negative bacterial pathogen;
wherein the Gram-negative bacterial pathogen is a serogroup O5 strain of P. aeruginosa,
the method comprising administering an effective amount of the monoclonal antibody of claim 6 to the patient.

14. A method of treating a patient,
wherein the patient is infected with a Gram-negative bacterial pathogen, or at risk of being infected with the Gram-negative bacterial pathogen;
wherein the Gram-negative bacterial pathogen is selected from the group consisting of P. aeruginosa, Burkholderia pseudomallei, Burkholderia thailandensis, Burkholderia cepacian, Burkholderia cenocepacia, Klebsiella pneumoniae, Serratia marcescens, Enterobacter cloacae, Salmonella enterica, Acinetobacter baumannii, Escherichia coli, and Bordetella pertussis,
comprising administering an effective amount of the monoclonal antibody of claim 4 to the patient.

15. A method of treating a patient,
wherein the patient is infected with a Gram-negative bacterial pathogen, or at risk of being infected with the Gram-negative bacterial pathogen;
wherein the Gram-negative bacterial pathogen is selected from the group consisting of P. aeruginosa, Burkholderia pseudomallei, Burkholderia thailandensis, Burkholderia cepacian, Burkholderia cenocepacia, Klebsiella pneumoniae, Serratia marcescens, Enterobacter cloacae, Salmonella enterica, Acinetobacter baumannii, Escherichia coli, and Bordetella pertussis,
comprising administering an effective amount of the monoclonal antibody of claim 7 to the patient.

16. An antibody against a Gram-negative bacterial pathogen, wherein:
the antibody is configured to bind to a flagellin protein of the Gram-negative bacterial pathogen; and
the flagellin protein has:
at least 38% identity with P. aeruginosa type b flagellin; and/or
at least 37% identity with B. pseudomallei flagellin.

17. The antibody of claim 16, wherein the antibody comprises a variable region with:
a heavy chain having a first CDR comprising residues 31-35 of SEQ. ID No. 10; a second CDR comprising residues 50-66 of SEQ. ID No. 10; and a third CDR comprising residues 99-112 of SEQ. ID No. 10; and
a light chain having a first CDR comprising residues 24-34 of SEQ. ID No. 12; a second CDR comprising residues 50-56 of SEQ. ID No. 12; and a third CDR comprising residues 89-97 of SEQ. ID No. 12.

18. The antibody of claim 17, wherein the antibody is a humanized antibody.


SEQUENCE LISTING (20 sequences)

SEQ ID NO: 1 - DNA, 616 bp, synthetic construct
  source 1..616: mol_type = other DNA
caacctancgggaagtagcccttgaccaggcatcccagagtcacagaggaaccagttgtatctccacacccaggggccagtggatagactgatgggggtgttgttttggctgaggagacggtgactgaggttccttgaccccagcagtccatagcgtagccactaccgtagccccctcttgcacagtaatagaccgcagagtcctcagatgtcaggctgcttagctgcatgtaggctgtgctggaggatgtgtctacagtcaatgtggccttgtccttgaacttttgaatgtagtgagtttcactatcagaagggtcaatattaccaatccattcaaggccttgtccaggcctctgcttcacccagtgcatccagtagttggtgaaggtgtagccagaagccttgcaggacagcttcactgaagccccaggcttcaccagctcagccccaggctgctgcagttggacctgggagtggacacctgtagctgctgctaccaaaaagaggatgatacagctccatcccatggtgagatcctgtgtgcttagtaactgtagagagaacagtgatcttatccccctgtactctccgttgatgaccccctgcgttataataa

SEQ ID NO: 2 - protein, 204 aa, synthetic construct
IITQGVINGEYRGIRSLFSLQLLSTQDLTMGWSCIILFLVAAATGVHSQVQLQQPGAELVKPGASVKLSCKASGYTFTNYWMHWVKQRPGQGLEWIGNIDPSDSETHYIQKFKDKATLTVDTSSSTAYMQLSSLTSEDSAVYYCARGGYGSGYAMDCWGQGTSVTVSSAKTTPPSVYPLAPGCGDTTGSSVTLGCLVKGYFPXG

SEQ ID NO: 3 - DNA, 558 bp, synthetic construct
  source 1..558: mol_type = other DNA
tttttaaacacgactgaggncctccagatgttaactgctcactggatggtgggaagatggatacagttggtgcagcatcagcccgttttatttccagcttggtcccccatccgaacgtatacgggacatgtgtaacttggaggcagaaataaactcccaaatcctcaccctccactctgctaatcttgagtgtgaaatctgtccctgaaccactgccactgaacctggctacgaccccacaaaatcggatggaaaccctgtaaatcaggagctgtggagactggcctggtttctggaggtaccagttcaaataggtgtttccattactgttttcaaggctctgactagacctgcaagagatggaggcttgatctccaagactgacaggcagggagagtggagtttgggtcatcacagcatcactgctggaagcaggaatccagaacaacagcaccaacaacctaacaggcaacttcattttgaggaggcaacctgaggagacagcctgaggagactgatcactccccatgtactctgcgttgataccactgcttaaaa

SEQ ID NO: 4 - protein, 185 aa, synthetic construct
LSSGINAEYMGSDQSPQAVSSGCLLKMKLPVRLLVLLFWIPASSSDAVMTQTPLSLPVSLGDQASISCRSSQSLENSNGNTYLNWYLQKPGQSPQLLIYRVSIRFCGVVARFSGSGSGTDFTLKISRVEGEDLGVYFCLQVTHVPYTFGWGTKLEIKRADAAPTVSIFPPSSEQLTSGXPQSCLK

SEQ ID NO: 5 - DNA, 611 bp, synthetic construct
  source 1..611: mol_type = other DNA
ccacctatgggaagtagcccttgaccaggcatcccagagtcacagaggaaccagttgtatctccacacccaggggccagtggatagactgatgggggtgttgttttggctgaggagacggtgactgaggttccttgaccccagcagtccatagcgtagccactaccgtagccccctcttgcacagtaatagaccgcagagtcctcagatgtcaggctgcttagctgcatgtaggctgtgctggaggatgtgtctacagtcaatgtggccttgtccttgaacttttgaatgtagtgagtttcactatcagaagggtcaatattaccaatccattcaaggccttgtccaggcctctgcttcacccagtgcatccagtagttggtgaaggtgtagccagaagccttgcaggacagcttcactgaagccccaggcttcaccagctcagccccaggctgctgcagttggacctgggagtggacacctgtagctgctgctaccaaaaagaggatgatacagctccatcccatggtgagatcctgtgtgcttagtaactgtagagagaacagtgatcttatccccctgtactctgcgttgataccactgcttataaaa

SEQ ID NO: 6 - protein, 201 aa, synthetic construct
FISSGINAEYRGIRSLFSLQLLSTQDLTMGWSCIILFLVAAATGVHSQVQLQQPGAELVKPGASVKLSCKASGYTFTNYWMHWVKQRPGQGLEWIGNIDPSDSETHYIQKFKDKATLTVDTSSSTAYMQLSSLTSEDSAVYYCARGGYGSGYAMDCWGQGTSVTVSSAKTTPPSVYPLAPGCGDTTGSSVTLGCLVKGYFP

SEQ ID NO: 7 - DNA, 558 bp, synthetic construct
  source 1..558: mol_type = other DNA
tcacttaaacccgactgaggncctccagatgttaactgctcactggatggtgggaagatggatacagttggtgcagcatcagcccgttttatttccagcttggtccccgatccgaacgtatacgggacatgtgtaacttggaggcagaaataaactcccaaatcctcagcctccactctgctaatcttgagtgtgaaatctgtccctgaaccactgccactgaacctgtctaggaccccacaaaatcggttggaaaccctgtaaatcaggagctgtggagactggcctggtttctggaggtaccagttcaaataggtgtttccattactgttttcaaggctctgactagacctgcaagagatggaggcttgatctccaagactgacaggcagggagagtggagtttgggtcatcacagcatcactgctggaagcaggaatccagaacaacagcaccaacaacctaacaggcaacttcattttgaggaggcaacctgaggagacagcctgaggagactgatcactccccatgtactctgcgttgataccactgattaaa

SEQ ID NO: 8 - protein, 185 aa, synthetic construct
LISGINAEYMGSDQSPQAVSSGCLLKMKLPVRLLVLLFWIPASSSDAVMTQTPLSLPVSLGDQASISCRSSQSLENSNGNTYLNWYLQKPGQSPQLLIYRVSNRFCGVLDRFSGSGSGTDFTLKISRVEAEDLGVYFCLQVTHVPYTFGSGTKLEIKRADAAPTVSIFPPSSEQLTSGXPQSGLS

SEQ ID NO: 9 - DNA, 475 bp, synthetic construct
  source 1..475: mol_type = other DNA
gaggtgcagctgcaggagtctggggctgagctggtaaagcctggggcttcagtgaagttgtcctgtaaggcttctggctacacttttaccagttactggatgcactgggtgaagcagaggcctggacaaggccttgagtggattggaatgattcgtcctaatagtggtagtacaaattataatgagaagttcaagagtaaggccacactgactgtagacaaatcctccagcacagcctacatgcaactcagcagcccgacatctgaggactctgcggtctattactgtgcaagaaggggctacggtagtagttatggatactatgctatggactactggggtcaaggaacctcagtcaccgtctccagcgctaaaacaacacccccatcagtctatccactggcccctgggtgtggagatacaactggttcctctgtgactctgggatgcctggtcaagggctacttccctgagt

SEQ ID NO: 10 - protein, 158 aa, synthetic construct
EVQLQESGAELVKPGASVKLSCKASGYTFTSYWMHWVKQRPGQGLEWIGMIRPNSGSTNYNEKFKSKATLTVDKSSSTAYMQLSSPTSEDSAVYYCARRGYGSSYGYYAMDYWGQGTSVTVSSAKTTPPSVYPLAPGCGDTTGSSVTLGCLVKGYFPE

SEQ ID NO: 11 - DNA, 350 bp, synthetic construct
  source 1..350: mol_type = other DNA
gaattcgcccttacgaattctgacattgtggtgacacagactacatcctccctgtctgcctctctgggagacagagtcaccatcagttgcagggcaagtcaggacattagcaattatctaaactggtatcagcagaaaccagatggaactgttaaactcctgatcaactacacatcaagattaaactcaggagtcccatcaaggttcagtggcagtgggtctggaacagattattctctcaccattagcaacctggagcaggaagatattgccacttacttttgccaacagggtaatacgcttccgtggacgttcggtggtggcaccaagctcgagataagggcgaattc

SEQ ID NO: 12 - protein, 105 aa, synthetic construct
DIVVTQTTSSLSASLGDRVTISCRASQDISNYLNWYQQKPDGTVKLLINYTSRLNSGVPSRFSGSGSGTDYSLTISNLEQEDIATYFCQQGNTLPWTFGGGTKLE

SEQ ID NO: 13 - protein, 123 aa, synthetic construct
QVQLVQSGAEVKKPGASVKVSCKASGYTFTSYWMHWVRQAPGQGLEWMGMIRPNSGSTNYNEKFKSRVTMTRDTSTSTAYMELSSLRSEDTAVYYCARRGYGSSYGYYAMDYWGQGTTVTVSS

SEQ ID NO: 14 - protein, 105 aa, synthetic construct
DIQMTQSPSSLSASVGDRVTITCRASQDISNYLNWYQQKPGKAPKLLIYYTSRLNSGVPSRFSGSGSGTDFTLTISSLQPEDFATYYCQQGNTLPWTQGTKVEIK

SEQ ID NO: 15 - protein, 120 aa, synthetic construct
QVQLVQSGAEVKKPGASVKLSCKASGYTFTNYWMHWVRQAPGQGLEWMGNIDPSDSETHYIQKFKDRVTLTADTSTSTAYMELSSLTSEDTAVYYCARGGYGSGYAMDCWGQGTTVTVSS

SEQ ID NO: 16 - protein, 112 aa, synthetic construct
DIVMTQTPLSLPVTLGQPASISCRSSQSLENSNGNTYLNWYLQKPGQSPQLLIYRVSIRFCGVPDRFSGSGSGTDFTLKISRVEAEDVGVYYCLQVTHVPYTFGQGTKLEIK

SEQ ID NO: 17 - protein, 120 aa, synthetic construct; identical to SEQ ID NO: 15

SEQ ID NO: 18 - protein, 112 aa, synthetic construct
DIVMTQTPLSLPVTPGQPASISCRSSQSLENSNGNTYLNWYLQKPGQSPQLLIYRVSNRFCGVPDRFSGSGSGTDFTLKISRVEAEDVGVYYCLQVTHVPYTFGQGTKLEIK

SEQ ID NO: 19 - protein, 129 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDPRGGYGSGANYYYYYGMDVWGQGTTVTVSS

SEQ ID NO: 20 - protein, 110 aa, Homo sapiens
DIQMTQSPSSLSASVGDRVTITCRASQSVRKYLNWYQQKPGKAPKLLIYASSLQSGVPSRFSGSGSGTDFTLTISSLQPEDFATYYCQQSNSTPWTFGQGTKVEIKRTVA